(19) United States
(12) Patent Application Publication
(10) Pub. No.: US 2020/0179295 A1
(43) Pub. Date: Jun. 11, 2020

(54) MRI-DETECTABLE MULTILAYER MICROCAPSULES FOR ULTRASOUND-TRIGGERED DELIVERY OF PHARMACOLOGICALLY ACTIVE AGENTS

(71) Applicants: THE UAB RESEARCH FOUNDATION, Birmingham, AL (US); THE BOARD OF TRUSTEES OF THE UNIVERSITY OF ALABAMA, Tuscaloosa, AL (US)

(72) Inventors: Eugenia Kharlampieva, Birmingham, AL (US); Veronika Kozlovskaya, Birmingham, AL (US); Jason Warram, Birmingham, AL (US); Mark Bolding, Hoover, AL (US); Yuping Bao, Tuscaloosa, AL (US)

(21) Appl. No.: 16/210,397

(22) Filed: Dec. 5, 2018

(51) Int. Cl. A61K 9/50; A61M 37/00; A61K 49/18; A61K 47/69

(52) U.S. Cl. CPC A61K 9/5073; A61M 37/0092; A61K 9/5089; A61K 49/1821; A61B 5/055; A61K 9/501; A61K 9/5026; A61K 9/5015; A61M 2037/0007; A61K 47/6925

(57) ABSTRACT

The theranostic biocompatible microcapsules provided are efficient contrast enhanced imaging agents that combine Magnetic Resonance Imaging (MRI) with ultrasound-triggered drug release for real-time tracking and targeted delivery in vivo. The capsules are assembled via layer-by-layer deposition of the natural polyphenol tannic acid and poly(N-vinylpyrrolidone) with iron oxide nanoparticles incorporated in the capsule wall. The nanoparticle-modified capsules exhibit enhanced T₁ and T₂ MRI contrast in a clinical MRI scanner. Loaded with the an anticancer drug such as doxorubicin the capsules circulate in the blood stream for at least 48 hours, an improvement compared to non-encapsulated nanoparticles. High-intensity focused ultrasound results in targeted drug release with a 16-fold increase in the pharmacologically active agent localization in tumors compared to off-target organs. Owing to the active contrast, long circulation, customizable size, shape, composition, and precise delivery of high payload concentrations, these materials present an improved platform for imaging-guided precision drug delivery.

### STATEMENT ON FUNDING PROVIDED BY THE U.S. GOVERNMENT

This invention was made with government support under Grant Nos.: NSF-DMR 1608728 and NSF-DMR 1149931 awarded by the U.S. National Science Foundation of the United States government. The government has certain rights in the invention.

### FIELD OF THE DISCLOSURE

The present disclosure is generally related to polymer and tannic acid layer-by layer capsules incorporating iron oxide nanoparticles. The present disclosure further relates to methods of manufacture and the use of the capsules for targeted delivery of a pharmacologically active agent released from the capsule by ultrasound exposure.

### BACKGROUND

Many cancers are heterogeneous diseases and current chemotherapeutic combinations elicit unwanted side effects arising from the non-specificity of drugs, limited bioavailability due to poor drug solubility, and dose-limiting toxicity (Allen & Cullis (2004) Science 303: 1818; Vrignaud et al., (2011) Biomaterials 32: 8593). Direct injection of drugs into the tumor site and systemic delivery of free drugs can be highly invasive and hinder the day-to-day life of the patient. A promising alternative, imaging-guided drug delivery, is non-invasive and allows more effective drug activity at tumor sites with reduced side effects (Huang & Jonathan (2017) Adv. Func. Mater. 27: 1603524; Phillips et al., (2014) Adv. Drug Delivery Rev. 76: 39; Funkhouser (2002) Curr. Drug Discov. 2: 17; Huang et al., (2016) Adv. Func. Mater. 26: 3818). This potential is fully realized when the delivery of drugs can be followed with common medical imaging protocols and precisely tailored to a specific spatial and temporal regimen. Therefore, the clinical advancement of more effective and life-saving cancer treatment strategies is coupled to the development of new drug carriers embodying features that lie beyond the capability of simple small molecule drugs.

In magnetic resonance imaging (MRI), the ‘gold standard’ for tumor imaging (Terreno et al., ((2010) Chem. Rev. 110: 3019; Lawrence et al., (2006) J. Clin. Oncol. 24: 1225), MRI pulse sequences can be adjusted to produce images that are “weighted” toward intrinsic T₁ or T₂ relaxation effects (Elster W. T. (1988) J. Comput. Assist. Tomogr. 12: 130). Small molecule contrast enhancement agents such as gadolinium-based chelates are commonly associated with T₁ contrast at normal doses (Elster et al., (1990) Radiology 174: 379). However, the high toxicity of uncoordinated Gd³⁺ requires each Gd³⁺ ion to be permanently associated with its chelating agent or carrier system to reduce tissue absorption of free ions (Rose: Jr & Choi (2015) Am. J. Med. 128: 943). Even so, sensitive patients may suffer adverse reactions and therefore cannot receive contrast-enhanced MRI (Bellin & Van Der Molen (2008) Eur. J. Radiol. 66: 160). This is because Gd-based agents have been associated with nephrogenic systemic fibrosis (Chopra et al., (2012) Int. J. Nephrol. 2012: 912189) and other health risks, and have been suggested for limited use by the World Health Organization and the U.S. FDA.

Conversely, although MRI-active ultra-small (less than 4 nm) iron oxide nanoparticles (NPs) are non-toxic, they tend to experience fast renal clearance and escape from blood circulation, limiting their use in disease tracking (Sherwood et al., (2017) Nanoscale 9: 11785; Longmire et al., (2008) Nanomedicine 3: 703). However, by incorporating iron oxide NPs into a protective polymeric drug carrier, MRI can be utilized to both visualize the target tissue and to monitor the path of the delivery agents without the drawbacks of toxicity incurred from lanthanide metals and fast clearance of unshielded NPs (Weinstein et al., (2009) J. Cereb. Blood Flow Metab. 30: 15). Iron oxide NPs can also provide additional benefit to polymer microencapsulation strategies as, since ultrasound (US)-induced mechanical force can facilitate the release of drugs from polymeric carriers (de Jong et al., (1991) Ultrasonics 29: 324) having the NPs embedded in the shell of the carrier. This improves the sensitivity to ultrasound via increasing the shell density (Skirtach et al., (2007) J. Mater. Chem. 17: 1050). A recent study has demonstrated an advanced look at the concept of “sonosensitizers” by showing the ultrasound-induced cavitation bubbles made possible by metal organic framework-derived carbon nanostructures (Pan et al., (2018) Adv. Mater. D01:10.1002/adma.201800180).

Ultrasound can provide controlled disruption of polymeric drug delivery particles as an advancing cancer treatment approach because it is completely non-invasive, inexpensive, available in any medical setting, and capable of applying mechanical force and/or thermal energy. Unlike other stimuli including pH/ionic strength change and enzymatic degradation, ultrasound offers precise control over spatio-temporal drug release and drug transport into solid tumors (Rizzitelli et al., (2015) J. Controlled Release 202: 21; Rychak & Klibanov (2014) Adv. Drug Delivery Rev. 72: 82; Milgroom et al., (2014) Colloids Surf, B. 116: 652). Furthermore, the ultrasound beam can be tuned to produce anywhere from mild pulses (less than 100 mWcm⁻²) useful for diagnostic imaging of sensitive organs, to hyperthermal irradiation (up to 10⁵ Wcm⁻²) used in ablative therapy of tumors (Kiessling et al., (2014) Adv. Drug Delivery Rev. 72: 15).

Owing to the enormous potential of grafting MRI and/or ultrasound-sensitive capability into a therapeutic agent, a number of systems have been developed to take advantage of one or both of these techniques, and in particular on micelles (Nasongkla et al., (2006) Gao: Nano Lett. 6: 2427; Wu et al., (2015): Langmuir 31: 7926; Vinh et al., (2015) Int. J. Nanomed. 10: 4137; Shiraishi et al., (2017) J. Controlled Release 253: 165) or polymer-containing nanoparticles (Huang et al., (2010) ACS Nano 4: 7151; Santra et al., (2012) ACS Nano 6: 7281; Hurley et al., (2016) Mol. Pharmaceutics 13: 2172; Chen et al., (2014) Chem. Mater. 26: 2105; Liang et al., (2015) Adv. Func. Mater. 25: 1451; Szczepanowicz et al., (2017) Colloids Surf. A 532: 351). For example, micelles made from Gd-DTPA/DACHPt (a platinum drug bound to the Gd chelator DTPA) conjugated to poly(ethylene glycol)-b-poly(glutamic acid) [PEG-b-P(Glu)] were shown to be useful for tracking treatment progress of a hepatocellular carcinoma using MRI (Vinh et al., (2015) Int. J. Nanomed. 10: 4137).

Iron oxide NPs coated with poly(vinylpyrrolidone) (PVPON) were used to enhance MR images of hepatic lesions, although many of the NP-PVPON preparations experienced highly attenuated contrast in vivo due to rapid clearance caused by a number of factors (Huang et al., (2010) ACS Nano 4: 7151). It has also been shown that mesoporous silica particles containing metalloporphyrins utilize ultrasound and MRI to enhance tissue treatment strategies (Huang et al., (2017) J. Am. Chem. Soc. 139: 1275).

To circumvent the clearance issues of coated nanoparticles and introduce the capability of drug loading, there have been reports of liposomes and synthetic polymer vesicles that are capable of MRI contrast (Qin et al., (2015) ACS Appl. Mater. Interfaces 7: 14043; Liu et al., (2015) Macromolecules 48: 739; Kim et al., (2016) Mol. Pharmaceutics 13: 1528). Liposomes with elastin-like peptides surrounding Gd-BOPTA were shown to release encapsulated doxorubicin in response to ultrasound-generated hyperthermia. However, these liposomes were also shown to release almost 20% of the loaded doxorubicin in only 30 min at a physiological temperature (Kim et al., (2016) Mol. Pharmaceutics 13: 1528). Additionally, liposomes are challenged by low stability in the bloodstream and must balance stabilization by hydrophilic polymers such as PEG with the ability to interact with their target (Sercombe et al., (2015) Front. Pharmacol. 6: 286).

In contrast to nanoparticles and micelles, layer-by-layer (LbL) assembled multilayer microcapsules provide much higher loading capacity and easily adjustable composition and properties (Gao et al., (2015) J. Mater. Chem. B 3: 1888). These hollow particles with narrowly dispersed size and shape are comprised of ultrathin multilayer shells (less than 50 nm) and micron-sized cavities and are assembled through LbL deposition of polymers onto sacrificial templates which are easily dissolved (Cui et al., (2014) Adv. Colloid Interface Sci. 207: 14; Koker et al., (2012) Chem. Soc. Rev. 41: 2867; Delcea et al., (2011) Adv. Drug Delivery Rev. 63: 730). Unlike other polymer vesicles, the chemical and physical properties of LbL capsules can be precisely tailored with minimal risk of destabilization by varying the polymer composition and number of layers in the shell (Mak et al., (2008) Chem. Mater. 20: 5475; Bedard et al., (2009) Soft Matter 5: 148). Both size and rigidity of these delivery vehicles can regulate the cellular internalization efficiency in a cell type-dependent way (Yan et al., (2103) Chem. Mater. 26: 452). Unlike rigid inorganic or polymeric nanoparticles that are excluded from cellular uptake at sizes greater than 150-200 nm, the upper size threshold for cell internalization of softer colloids by non-phagocytotic cells has been shown to be much higher where softer hollow capsules and flexible hydrogel particulates with sizes up to 3-5 μm could be internalized by cells due to their high elasticity and squeezeability (Gratton et al., (2008) Proc. Natl. Acad. Sci. U.S.A. 105: 11613; Shimoni et al., (2012) ACS Nano 7: 522; Kozlovskaya et al., (2014) ACS Nano 8: 5725; Xue et al., (2015) ACS Applied Mater. & Interfaces 7: 13633; Alexander et al., (2015) Adv. Healthcare Mater. 4: 2657). Moreover, unlike rigid delivery vehicles that suffer from rapid blood clearance (Neuberger et al., (2005) J. Magnetism and Magnetic Mater. 293: 483), tissue toxicity (Rose Jr & Choi (2015) Am. J. Med. 128: 943), and activation of complex immune response, soft polymer vehicles, such as hollow polymer nanothin capsules are advantageous because of their mechanical integrity (unlike micelles or liposomes), much higher drug loading capacity, and due to shielding of drugs from immunologic clearance (Chiu et al., (2016) ACS Appl. Mater. & Interfaces 8: 18722; Johnston et al., (2012) ACS Nano 6: 6667).

There are very few reports of NP-modified microcapsules with MRI contrast capability. The examples include recent studies showing that electrostatically-assembled poly(styrene sulfonate)/poly(allylamine hydrochloride) (PSS/PAH) and poly(L-arginine hydrochloride)/dextran sulfate (Parg/DS) capsules can be modified with iron oxide NPs and produce MRI contrast rivaling that of Gd (Li et al., (2013) BioNanoScience 4: 59; Abbasi et al., (2011) J. Phys. Chem. 115: 6257; German et al., (2016) Phys. Chem. Chem. Phys. 18: 32238). Additionally, metal phenolic networks of tannic acid (TA) and Gd³⁺, Fe³⁺, or Mn²⁺ ions that could be assembled into microcapsules with MRI capability have been reported (Guo et al., (2014) Angew. Chem. Int. Ed. 53: 5546) TA is a natural antioxidant able to participate not just in metal coordination (Ejima et al., (2013) Science 341, 154-157; Guo et al., (2016) Nat. Nano 11: 1105-1111) but also ionic pairing (Shutava et al., (2005) Macromolecules 38: 2850-2858) and hydrogen bonding (Zhuk et al., (2014) ACS Nano 8: 7733-7745; Kozlovskaya et al., (2008) Soft Matter 6: 3596-3608; Erel-Unal & Sukhishvili (2008) Macromolecules 41: 3962-3970).

Although current reported systems show MRI-sensitivity, drug loading and release have not been demonstrated in those reports. Several studies showed that multilayered PSS/PAH microcapsules with ZnO, Fe₃O₄, or silica infused in the shell could be destroyed via sonication (Timin et al., (2017) Part. Part. Syst. Charact. 34: 1600417; Kolesnikova et al., (2010) Adv. Func. Mater. 20: 1189; Korolovych et al., (2016) Phys. Chem. Chem. Phys. 18: 2389; Shchukin et al., (2006) Langmuir 22: 7400). However, there have been no reports of an LbL microcapsule system that demonstrates both MRI contrast and ultrasound-triggered in vivo drug release in one study.

It has been shown that biocompatible microcapsules of hydrogen-bonded (TA/PVPON) have antioxidant, immunomodulatory, and cytoprotective properties (Kozlovskaya et al., (2012) Adv. Func. Mater. 22: 3389; Kozlovskaya (2015) Adv. Healthcare. Mater. 4: 686; Chen et al., (2013) Biomacromolecules 14: 3830) and can be used for long-term storage of doxorubicin (DOX) (Liu et al., (2014) Soft Matter 10: 9237). TA/PVPON microcapsules can deliver encapsulated DOX under both low-intensity diagnostic (power intensities of 0.1 Wcm⁻²) and high-intensity therapeutic (>10 Wcm⁻²) ultrasound irradiation (Chen et al., (2017) ACS Nano 11: 3135). We showed that the ultrasound application time and acoustic power could easily be manipulated to tune the release of DOX from DOX-loaded (TA/PVPON) capsules (Chen et al., (2017) ACS Nano 11: 3135).

What is needed, therefore, is a drug carrier that combines the diagnostic potential of magnetic resonance imaging (MRI) visualization with the precision of ultrasound-controlled release of the encapsulated drug could significantly advance the field of cancer treatment as it represents an ideal balance between maximizing the utilization of clinically available technology and simplicity without compromising drug efficacy or patient health.

### SUMMARY

The present disclosure provides compositions comprising iron oxide impregnated layer-by-layer polymer capsule walls encapsulating at least one pharmacologically active agent desired to be delivered to a target site within patient. Further provided are methods of making and using these compositions to provide for the MRI monitoring of the progress of delivery of a pharmacologically active agent to a selected site within the patient. Once the composition has been concentrated to a desired amount at the target site, the application of an ultrasound emission can disrupt the integrity of the layer-by-layer polymer capsule walls to release the pharmacologically active agent.

Briefly described therefore, one aspect of the present disclosure, therefore, encompasses embodiments of a composition comprising a layer-by-layer plurality of polymer bilayers, wherein each polymer bilayer can comprise a polymer layer hydrogen-bonded to a polyphenolic tannin layer, and wherein at least one of the bilayers can further comprise a plurality of iron oxide nanoparticles attached thereto.

In some embodiments of this aspect of the disclosure, the composition can comprise from 1 to about 20 polymer bilayers.

In some embodiments of this aspect of the disclosure, the composition can comprise 6 polymer bilayers.

In some embodiments of this aspect of the disclosure, the polymer layer of each bilayer can be a poly(N-vinylpyrrolidone) layer.

In some embodiments of this aspect of the disclosure, the iron oxide nanoparticles can comprise ferric oxide and tannic acid.

In some embodiments of this aspect of the disclosure, the plurality of iron oxide nanoparticles can be attached to at least one polymer layer.

In some embodiments of this aspect of the disclosure, the at least one polymer layer having the iron oxide nanoparticles attached thereto can be a poly(N-vinylpyrrolidone) layer.

In some embodiments of this aspect of the disclosure, the composition can be a capsule defining an internal volume.

In some embodiments of this aspect of the disclosure, the layer-by-layer composition is deposited as a capsule encapsulating a solid core substrate.

In some embodiments of this aspect of the disclosure, the core substrate is in contact with a polyphenolic tannic acid layer of a bilayer.

In some embodiments of this aspect of the disclosure, the composition can further comprise a plurality of poly(N-vinylpyrrolidone) layers, each of said poly(N-vinylpyrrolidone) layers alternating with a layer of iron oxide-tannic acid nanoparticles.

In some embodiments of this aspect of the disclosure, the composition can further comprise an outer poly(N-vinylpyrrolidone) layer encapsulating the layer-by-layer composition.

In some embodiments of this aspect of the disclosure, the outer poly(N-vinylpyrrolidone) layer encapsulating the layer-by-layer composition can comprise a functional moiety attached thereto.

In some embodiments of this aspect of the disclosure, the outer poly(N-vinylpyrrolidone) layer encapsulating the layer-by-layer composition can comprise a functional moiety attached thereto, the functional moiety being selected from the group consisting of: a detectable moiety, an immunomodulatory molecule, a growth factor, a cell receptor ligand, a polypeptide cell receptor, or any combination thereof.

In some embodiments of this aspect of the disclosure, the core substrate can comprise at least one pharmacologically active agent.

In some embodiments of this aspect of the disclosure, the composition can encapsulate at least one pharmacologically active agent within the internal volume.

In some embodiments of this aspect of the disclosure, the core substrate can be removable.

Another aspect of the disclosure encompasses embodiments of a capsule, wherein the capsule can comprise a wall encapsulating a pharmacologically active agent, wherein the wall of the capsule can comprise: a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer; a plurality of poly(N-vinylpyrrolidone) layers, each of said poly(N-vinylpyrrolidone) layers alternating with a layer of iron oxide-tannic acid nanoparticles; and an outer poly(N-vinylpyrrolidone) layer.

In some embodiments of this aspect of the disclosure, the outer poly(N-vinylpyrrolidone) layer can comprises a functional moiety attached thereto.

In some embodiments of this aspect of the disclosure, the outer poly(N-vinylpyrrolidone) layer can comprises a functional moiety attached thereto, the functional moiety being selected from the group consisting of: a detectable moiety, an immunomodulatory molecule, a growth factor, a cell receptor ligand, a polypeptide cell receptor, or any combination thereof.

In some embodiments of this aspect of the disclosure, the capsule is mixed with a pharmaceutically acceptable carrier.

Still another aspect of the disclosure encompasses embodiments of a method of generating a layer-by layer composition, wherein said layer-by layer composition comprises an MRI contrast agent and a pharmacologically active composition, the method comprising the steps of: (a) obtaining a core substrate particle comprising a pharmacologically active agent; (b) obtaining a population of tannic acid-modified iron-oxide nanoparticles; (c) contacting the porous silica core of step (a) with a solution of a cationic polymer, thereby coating the porous silica core particle with the cationic polymer; (d) encapsulating the porous silica core particle of step (c) by depositing thereon a capsule comprising a layer-by-layer polymer coating, wherein said polymer coating comprises a plurality of tannic acid-poly(N-vinylpyrrolidone)-bilayers, wherein the tannic acid layer of a first bilayer is in contact with the porous silica core; (e) depositing a plurality of tannic acid-modified iron-oxide nanoparticles on a poly(N-vinylpyrrolidone) layer of a bilayer; (f) depositing a plurality of alternating poly(N-vinylpyrrolidone)-tannic acid-modified iron-oxide nanoparticle layers on the surface of the product of step (e); (g) depositing an outer poly(N-vinylpyrrolidone) layer on the surface of the product of step (f); and (h) removing the silica core from the capsule while leaving the pharmacologically active agent within the capsule.

In some embodiments of this aspect of the disclosure, the method can further comprise the step of attaching a functional moiety to the outer poly(N-vinylpyrrolidone) layer, the functional moiety being selected from the group consisting of: a detectable moiety, an immunomodulatory molecule, a growth factor, a cell receptor ligand, a polypeptide cell receptor, or any combination thereof.

Yet another aspect of the disclosure encompasses embodiments of a method of delivering a pharmacologically active agent to a patient in need thereof, the method comprising the steps: (a) administering to a patient a pharmacologically active composition comprising a capsule, wherein the capsule comprises a wall encapsulating a pharmacologically active agent, wherein the wall of the capsule comprises: a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer; a plurality of poly(N-vinylpyrrolidone) layers, each of said poly(N-vinylpyrrolidone) layers alternating with a layer of iron oxide-tannic acid nanoparticles; and an outer poly(N-vinylpyrrolidone) layer; (b) monitoring by magnetic resonance imaging (MRI) the delivery of the pharmacologically active composition to a selected site within the patient; and (c) administering an ultrasound emission to the patient, wherein the ultrasound emission has a frequency and intensity that disrupts the wall of the capsule of the pharmacologically active composition within the patient, thereby releasing the pharmacologically active agent to a tissue of the selected site patient.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates the multilayer assembly of TA and PVPON polymer layers on Doxorubicin-loaded porous 3 μm SiO₂ microparticles followed by dissolution of the core to obtain hollow (TA/PVPON)ₙ capsules, with n denoting the number of bilayers. 4 nm TA-modified Fe₂O₃ nanoparticles (NPs) are deposited in alternating layers with PVPON after core dissolution to form NP-modified capsules with loaded Doxorubicin. The final shell composition is (TA/PVPON)₆(Fe₂O₃/PVPON)₂.

FIG. 2A illustrates a photograph of the (TA/PVPON)₆ (left) and (TA/PVPON)₆(Fe₂O₃/PVPON)₂ (right) capsule pellets at the bottom of Eppendorf tubes.

FIGS. 2B-2C illustrate SEM images of (TA/PVPON)₆(Fe₂O₃/PVPON)₂ capsules.

FIG. 2D illustrates an AFM image of (TA/PVPON)₆(Fe₂O₃/PVPON)₂ capsules.

FIGS. 2F and 2E illustrate TEM images of (TA/PVPON)₆ (FIG. 2F) and (TA/PVPON)₆(Fe₂O₃/PVPON)₂ (FIG. 2F) capsules.

FIG. 3A illustrates CLSM images of (Panels (a)-(e)) (TA/PVPON)₈ and (Panels (f)-(j)) (TA/PVPON)₆(Fe2O3/PVPON)₂ capsules after incubation with FITC-dextran fluorescent probes with molecular weights of (Panels (a) and (f)) 250 kDa, (Panels (b) and (g)) 70 kDa, (Panels (c) and (h)) 20 kDa, (Panels (d) and (i)) 4 kDa and (Panels (e) and (j)) with Alexa Fluor 488 fluorescent dye, MW 580 Da.

FIG. 3B illustrates the dependence of capsule permeability (%) on the molecular weight of the fluorescent probe after 15 min of exposure.

FIGS. 3C and 3D illustrate CLSM images of (TA/PVPON)₆(Fe₂O₃/PVPON)₂ capsules before (FIG. 3C) and after low intensity diagnostic ultrasound (2.25 MHz; 115 mWcm−2) for 15 min (FIG. 3D).

FIG. 3E illustrates DOX release from DOX-(TA/PVPON)₆(Fe₂O₃/PVPON)₂ capsules (0.8 pg DOX per capsule) after therapeutic ultrasound treatment (20 kHz, 14 Wcm−2, 60 s in 20 s bursts with 20 s rests) at pH=7.4 as compared to non-treated. Scale bar is 5 μm in all CLSM images. Data is presented as mean±SD in all plots with bars or markers representing the mean and vertical lines representing the SD. Sample size (n)=150 (50 capsules in 3 locations) for all measurements in plot (FIG. 3B) while n=3 for each measurement in plot. The probability (P) values (***p<0.0001) shown in (FIG. 3B) are the result of unpaired, two-tailed T-tests given the mean, SD, and n values and “ns” designates no statistically significant difference.

FIGS. 4A and 4B illustrate T₁ (FIG. 4A) and T₂ (FIG. 4B) relaxation curves for (TA/PVPON)₆ capsules with NPs deposited from increasing solution concentrations. 3 T MRI T₁-weighted (TE 11; TR 500) (FIG. 4C, top panel) and T2-weighted (TE 81; TR 4000) (FIG. 4C, bottom panel) images of (TA/PVPON)₆(Fe₂O₃/PVPON)₂ capsules above the commercial contrast agent ProHance (gadoteridol). The buffer solution and NP-free capsules were placed in between the capsule and Gd rows and are labeled accordingly. The corresponding concentrations of iron and gadolinium are listed above and below the images, respectively.

FIGS. 5A-5C illustrate 3 T MRI images of mice injected with the DOX-(TA/PVPON)₆(Fe₂O₃/PVPON)₂ capsules (1×10⁸ capsules mL−1) after 5 min (FIG. 5A) and 4 h (FIG. 5B) (top image: T₁-weighted). A mouse 48 h after injection of the same capsules (c; right) and a control capsule-free mouse after 48 h (FIG. 5C; left) (bottom image: T₂-weighted).

FIG. 6 is a schematic representation of in vivo administration of DOX-loaded nanoparticle-modified (NP) capsules: DOX-(TA/PVPON)₆(Fe₂O₃/PVPON)₂. The capsules are capable of both in vivo imaging and ultrasound-triggered drug delivery: athymic nude female mice were injected with MDA-MB-231 triple negative breast cancer cells and allowed to grow bilateral flank tumors. The mice were injected with 2×10⁸ capsules mL−1 and 30 μL/kg definity microbubbles during simultaneous treatment with 1.0 MHz HIFU (750 mVp/p 10 ms bursts; 1 Hz repetition rate (1% duty cycle); 120 s).

FIGS. 7A-7C illustrate tumors from four mice (m1-m4) imaged by an IVIS III Lumina bioluminescence imager in (FIG. 7A, column (c)) brightfield and (FIG. 7A, columns (b) and (d)) fluorescence modes with quantified DOX fluorescence (FIG. 7B) in ultrasound-treated (+US) and non-treated (−US) tumors (15 min post-injection; 2×10⁸ capsules mL−1 in the tail vein; high intensity focused ultrasound to one of the two flank tumors). (FIG. 7C) Concentration of DOX in tumor lysates (ng mL−1) and off-target organs as measured by HPLC-MS. Data is presented as mean±SD in all plots with bars representing the mean and vertical lines representing the SD. Sample size (n)=6313 and 7255 pixel counts in plot (FIG. 7B) for untreated (−US) and ultrasound-treated (+US) tumor fluorescence images, respectively, while n=4 for each tissue measurement in plot (FIG. 7C). The probability (P) values shown in both plots are the result of unpaired, two-tailed t-tests given the mean, SD, and n values and “ns” designates no statistically significant difference.

FIG. 8A illustrates histology images of (Panels (a) and (c)) the control tumor (−US) and (Panels (b) and (d)) the ultrasound-treated tumor (+ultrasound) tissues from mouse 1 (m1) and mouse 2 (m2) showing fluorescence from DOX.

FIG. 8B is a graph illustrating the quantification of iron present in control (−US-tumors) and ultrasound-treated (+US tumors) tumors from four mice that received 2×10⁸ capsule mL−1 tail vein injections as measured by relaxometry. Scale bar is 100 μm in all images. Data is presented as mean±SD with bars representing the mean and vertical lines representing the SD. Sample size (n)=4 for each measurement for untreated (−US) and ultrasound-treated (+US) tumor samples, respectively. Unpaired, two-tailed t-tests given the mean, SD, and n values showed no statistically significant difference, represented by “ns”.

FIG. 9 illustrates 3.0 T MRI images of the (TA/PVPON)₆(Fe₂O₃/PVPON)₂ capsule suspensions (left: T₁-weighted; right: T₂-weighted) without fetal bovine serum (I), and in the presence of 100% FBS (II) imaged after 4 h (Row (a)) and 24 h (Row (b)) of incubation at 37° C.

FIG. 10 illustrates 3.0 T MRI image (T₂-weighted) of the (TA/PVPON)₆(Fe₂O₃/PVPON)₂ capsule suspensions before (bottom right) and after (top) US treatment. ROI analysis shows the average intensity and standard deviation. The average change in contrast intensity over 3 slices was 8.0±1.8%. Mean and SD are shown in the ROI analysis, with n=70-80 pixel counts in each image. Unpaired, two-tailed T-tests give a P value <0.0001 for the change in contrast intensity.

FIG. 11 illustrates 9.4 T images (T₁-weighted) of the bilateral flank tumors in mouse models. (Panel (a)) Shows the uptake of the (TA/PVPON)₆(Fe₂O₃/PVPON)₂capsules as brightness in the round core of the tumors while (Panel (b)) shows the control tumors (saline injection) without the characteristic imaging contrast. Arrows point to the tumors in both images.

FIG. 12 illustrates 3.0 T MR images of mice with ROIs drawn over the kidney at time points of 5 min and 4 h (top), and ROIs over the heart 48 h (bottom) after injection of the capsules. Top image is T₁ weighted and bottom image is T₂ weighted. The mean intensity and SD are shown along with the pixel count (n; area in pix²) for each ROI. Unpaired, two-tailed t-tests comparing the two ROIs in each image resulted in a P value <0.0001.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### ABBREVIATIONS

LbL, layer-by-layer; PVPON, poly(N-vinylpyrrolidone); TA, tannic acid; TEM, transmission electron microscope; FITC, fluorescein isothiocyanate; US, ultrasound; MRI, magnetic resonance imaging; PEI, polyethylenimine;

### DEFINITIONS

In describing and claiming the disclosed subject matter, the following terminology will be used in accordance with the definitions set forth below.

The terms “administering” and “administration” as used herein refer to introducing a composition of the present disclosure into a subject.

The term “antibody” as used herein refers to polyclonal and monoclonal antibody preparations, as well as preparations including hybrid antibodies, altered antibodies, F(ab′)₂ fragments, F(ab) fragments, Fv fragments, single domain antibodies, chimeric antibodies, humanized antibodies, and functional fragments thereof which exhibit immunological binding properties of the parent antibody molecule.

The term “capsule” as used herein refers to a hollow structure wherein an internal volume is defined by an outer shell comprising a layer-by-layer composition according to the disclosure, wherein the layer-by-layer comprises bilayers consisting of PVPON and TA and wherein at least one PVPON has ferric oxide nanoparticle moieties attached thereto. While the defined internal volume may be occupied by a core on which the layer-by-layer composition is formed, the internal volume may be voided on contents such as when the core former is removed, whereupon the volume may receive, for example an amount of a pharmacologically active agent.

The term “cell” as used herein refers to any natural or artificial cell, animal, plant, bacterial, or a viral particle that be viable or dead. Such cells may be isolated from an animal or human subject or tissue thereof, or a cultured cell previously isolated from a subject source. An artificial cell includes, but is not limited to, an artificially engineered entity derived from such as a unicellular microorganism wherein all or some of the genetic material has been replaced.

The term “coating” as used herein refers to a multilayered coating encapsulating a core structure such as, but not limited to a removable silica core, a nanoparticle, a pharmacologically active composition, or the like. The coating may also be applied to a surface of other than a core such as, but not limited to, a substantially planar surface such as a silica wafer, and the like. In such a coating or coat of the present disclosure, a first layer or coat can comprise a polymer or units thereof that can be hydrogen-bonded to a substrate surface or to an outer cell membrane surface and, while thus bonded to a cell or cell aggregate does not significantly reduce the viability, physiology, or functioning of the cell type (for example, by retaining responsiveness to glucose in the case of coated pancreatic islets). In embodiments of the compositions of the disclosure the first layer can be, but is not limited to, poly(N-vinylpyrrolidone).

The term “functional moiety” as used herein refers to any molecule that may be attached to the outer surface of the outermost layer of the embodiments of the bilayer coatings of the disclosure. It is contemplated, but not intended to be limiting, for such moieties to be an imaging moiety (including a fluorescent dye, radiolabel, and the like), an immunomodulatory molecule, a growth factor, or any combination thereof, and the like.

The term “growth factor” as used herein refers to a peptide or polypeptide that can be, but is not limited to, a ligand that specially binds to a polypeptide or other receptor of a cell and includes, but is not limited to, a Acrp30, adipocytes complement related protein 30 kDa (adiponectin); ALCAM, activated leukocyte cell adhesion molecule; BDNF, brain-derived neurotrophic factor; BLC, B-lymphocyte chemoattractant; BMP, bone morphogenetic protein; BTC, β-cellulin; CCR, CC-chemokine receptor; CLC, cardiotrophin-like cytokine; CV, coefficient of variance; CXCR, CXC-chemokine receptor; DAB, 3,3′-diaminobenzidine; DAN, differential screening-selected gene aberrative in neuroblastoma; ECL, enhanced chemiluminescence; EDG-1, estrogen down-regulated gene 1; EGF, epidermal growth factor; ELISA, enzyme-linked immunosorbant assay; ET-1, endothelin 1; ETAR, endothelin receptor type A; FGF, fibroblast growth factor; GDF, growth and differentiation factor; GFR, Glial cell line-derived neurotrophic factor receptor; HB-EGF, heparin-binding EGF-like factor; HCC, hemofiltrate CC chemokine; ICAM, intercellular adhesion molecule; IFN, interferon; IGF, insulin-like growth factor; IGFBP, insulin-like growth factor binding protein; IgG, immunoglobulin gamma; IL, interleukin; I-TAC, Interferon-inducible T-cell alpha chemoattractant; LCK, lymphocyte cell-specific protein-tyrosine kinase; LIF, leukemia inhibitory factor; MCP, monocytes chemoattractant protein; M-CSF, macrophage colony stimulating factor; MIP, macrophage inflammatory protein; MMP, matrix metalloproteinase; MSP, macrophage stimulating protein; NAP, neural antiproliferation factor; NGF, nerve growth factor; NRG, neuregulin; NT, neurotensin; PDGF, platelet-derived growth factor; PIGF, placental growth factor; SCF, stem cell factor; TARC, thymus- and activation-regulated chemokine; TGF, transforming growth factor; TIMP, tissue inhibitor of metalloproteinases; TNF, tumor necrosis factor; TNFRSF, TNF receptor superfamily member; TNFSF, TNF superfamily member; TRAIL, TNF-related apoptosis inducing ligand; TRANCE, tumor necrosis factor-related activation induced cytokine; uPAR, urokinase plasminogen activator receptor; VCAM, vascular cellular adhesion molecule; VEGF, vascular endothelial growth factor.

The term “imaging agent” as used herein refers to a labeling moiety that is useful for providing an indication of the position of the label and adherents thereto, in a cell or tissue of an animal or human subject, or a cell or tissue under in vitro conditions. Such agents may include those that provide detectable signals such as fluorescence, luminescence, radioactivity, or can be detected by such as magnetic resonance imaging.

The term “immunomodulatory” as used herein refers to the generic modulation (i.e. not immunogenic per se) of the immune response in a desired fashion.

The term “label” or “tag” as used herein refers to a molecule that, when appended by, for example, without limitation, covalent bonding or hybridization to another moiety, for example, also without limitation, a nanoparticle provides or enhances a means of detecting the other moiety. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal-generation detection methods include: chemiluminescence, electrochemiluminescence, Raman, colorimetric, hybridization protection assay, and mass spectrometry. Radionuclides may be either pharmacologically active or diagnostic; diagnostic imaging using such nuclides is also well known. Typical diagnostic radionuclides include, but are not limited to, ⁹⁹Tc, ⁹⁵Tc, ¹¹¹In, ⁶²Cu, ⁶⁴Cu, ⁶⁷Ga, ⁶⁸Ga.

The term “layer-by-layer (LbL) assembly” as used herein refers to a technique for surface coating that depends on the controllable adsorption of two or more species on a surface through certain type of interactions (Decher & Hong (1991) Makromolekulare Chemie-Macromolecular Symposia 46: 321; Decher, G. (1997) Science 277: 1232). It has almost no restrictions on the type of interactions between the building blocks (Kharlampieva et al., (2009) Advanced Mats 21: 3053; Xu et al., (2007) Polymer 48: 1711) from conventional electrostatic forces to unconventional host-guest interactions, or covalent bonding. Further, it can accommodate different types of building blocks (Kharlampieva et al., (2009) Advanced Mats 21: 3053; Xu et al., (2007) Polymer 48: 1711) such as small molecules, polymers, bio-macromolecules and nanoparticles on a variety of types and shapes of surface templates (Kharlampieva et al., (2009) Advanced Mats 21: 3053). The most attractive property of LbL assembly is the well-defined structure of the coatings with controllable and predictable thickness growth from nanometer to millimeter scale (Kharlampieva et al., (2009) Advanced Mats 21: 3053; Xu et al., (2007) Polymer 48: 1711; Quinn et al., (2007) Chem. Soc. Revs. 36: 707; Such et al., (2011) Chem. Soc. Revs. 40: 19).

The term “multilayered composition” as used herein refers to a layer-by-layer-formed structure of superimposed polymer layers. The layers can be alternating PVPON and TA layers that bond by hydrogen bonds. In the coatings of the disclosure, at least one of the TA layers is modified by having ferric oxide nanoparticles attached thereto (a Fe₂O₃-TA layer). In some embodiments, the Fe₂O₃-TA layer can be embedded within the multilayered composition, thereby having a PVPON layer on each side of the Fe₂O₃-TA layer. In other embodiments, the Fe₂O₃-TA layer is disposed on one surface of the PVPON-TA bilayer.

The term “nanoparticle” as used herein refers to a particle having a diameter of between about 1 and about 1000 nm, preferably between about 100 nm and 1000 nm, and most preferably between about 50 nm and 700 nm. Similarly, by the term “nanoparticles” is meant a plurality of particles having an average diameter of between about 50 and about 1000 nm. The term “nanoparticle” as used herein may refer to a core component encapsulated by a layer-by layer coating according to the disclosure or to a capsule formed from a Fe₂O₃-TA-PVPON layer-by layer coating composition of the disclosure. The term “nanoparticle” may also refer to such as a ferric oxide nanoparticle that may be attached to a tannic acid layer.

The term “oncolytic virus” as used herein refers to a virus that can selectively kill neoplastic cells. Killing of the neoplastic cells can be detected by any method established in the art, such as determining viable cell count, cytopathic effect, apoptosis of die neoplastic cells, synthesis of viral proteins in the neoplastic cells (e.g., by metabolic labeling, Western analysis of viral proteins, or reverse transcription polymerase chain reaction of viral genes necessary for replication), or reduction in size of a tumor.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a diluent, adjuvant, excipient, or vehicle with which a layer-by layer capsule of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the layer-by layer capsule and pharmaceutically acceptable carriers can be sterile. Water is a useful carrier when the layer-by layer capsule is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The pharmaceutical compositions of the subject invention can be formulated according to known methods for preparing pharmaceutically useful compositions. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmacologically active agent” as used herein, refers to any agent, such as a drug, capable of having a physiologic effect (e.g., a therapeutic or prophylactic effect) on eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides including but not limited to, antibodies, and polynucleotides, and a biologic such as oncolytic viruses.

The pharmacologically active agent can be selected from a variety of known classes of drugs, including, for example, analgesics, anesthetics, anti-inflammatory agents, antihelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

The term “polyethylenimine (PEI)” as used herein refers to a polymer with repeating unit composed of the amine group and two carbon aliphatic CH₂CH₂ spacer. Linear polyethyleneimines contain all secondary amines, in contrast to branched PEIs which contain primary, secondary and tertiary amino groups. Totally branched, dendrimeric forms were also reported.

The term “polymer” as used herein refers to molecules comprising two or more monomer subunits that may be identical repeating subunits or different repeating subunits. A monomer generally comprises a simple structure, low-molecular weight molecule containing carbon. Polymers may optionally be substituted. A preferred polymer of the disclosure is polyvinylpyrrolidone.

The term “polymer bilayer” as used herein refers to a first layer of poly(N-vinylpyrrolidone) and a layer of a polyphenol (tannic acid) hydrogen-bonded thereto. In embodiments where the bilayers encapsulate a cell or aggregate of cells, it is preferred that the layer being proximal to the underlying cell or cells is poly(N-vinylpyrrolidone). In such embodiments, the outermost biocompatible layer, not having a polyphenol layer thereon, may be derivatized for the attachment of such as a labeling moiety, or other functional moiety. The coatings of the disclosure further include at least one TA layer wherein some or all of the TA monomer units have conjugated thereon on or more ferric oxide nanoparticles. The resulting Fe₂O₃-TA layer(s) may be located as the inner most layer of the capsule structure that is proximal to an encapsulated volume, sandwiched within non-Fe₂O₃ nanoparticle-containing bilayers. It is also contemplated that a polymer bilayer according to the disclosure may have as the outermost layer a PVPON polymer layer that may be further modified by the attachment thereto of other functional moieties as herein disclosed.

The term “polyphenol” as used herein refers to structural class of natural, synthetic and semi-synthetic organic chemicals characterized by the presence of large multiples of phenol units generally moderately water-soluble compounds, with molecular weight of 500-4000 Da, at least 12 phenolic hydroxyl groups, and 5-7 aromatic rings per 1000 Da, where the limits to these ranges are necessarily somewhat flexible, and include, but are not limited to the tannins.

The term “(PVPON/TA)ₙPVPON” as used herein refers to a multi-layered composition such as, but not limited to a coating of a silica surface, a cell, or to plurality of cells according to the present disclosure, the coating comprising “n” layers. The designator “n” denotes the number of bilayers on the multi-layered coating, “n” ranging from at least one to about 10. In embodiments where “n” is 1.5, 2.5, 3.5, 4.5, and the like, the 0.5 denotes that the multi-layered coating has an outer layer of poly(N-vinylpyrrolidone) not having a polyphenol (e.g. tannic acid) layer disposed thereon.

The term “subject” or “patient” as used herein means both mammals and non-mammals. Mammals include, for example, humans; non-human primates, e.g. apes and monkeys; and non-primates, e.g. dogs, cats, cattle, horses, sheep, and goats.

The terms “support surface” and “core support” as used herein refers a surface receiving a layer-by-layer composition according to the disclosure. In some embodiments, the support surface is that of a silica core that may be removed from the layer-by-layer construct to leave a volume or space encapsulated by a capsule. In some other embodiments, the support surface can be a substantially planar surface such as, but not limited to a silica or glass wafer on which the layer-by-layer composition of the disclosure is deposited. Most advantageously, a silica core is porous, allowing a pharmacologically active agent or agents in solution to permeate the core substrate. Once the core substrate has been encapsulated by the layer-by-layer compositions of the disclosure, the silica material may be removed by the use of such as hydrofluoric acid (at a concentration and/or for a time consistent with preserving the pharmacologically active agent) leaving the pharmacologically active agent encapsulated within the layer-by-layer capsule wall.

The terms “treating” or “treatment” as used herein refer to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

The term “volume” as used herein refers to a space that is defined by a layer-by-layer capsule. The layer of a polymer bilayer is closest to the volume or the contents contained therein is the “proximal” layer whereas the polymer layer the furthest from the volume (space) or the contents contained therein is the “distal” layer.

In describing and claiming the disclosed subject matter, the following terminology will be used in accordance with the definitions set forth below.

### DESCRIPTION

The disclosure provides embodiments of a composition, and methods of their use, that allow the regulated delivery of pharmacologically active agents to a location such as the site of a tumor and also act as an MRI contrast agent for imaging the same location. The composition of the disclosure comprise a hybrid iron oxide NP-(TA/PVPON) multilayer vehicle for the targeted delivery of pharmacologically active agents, and in particular anticancer agents. Ultrasound can be used as an external trigger for the drug release from a biocompatible MR-visible polymeric shell.

Intravenous administration of free iron oxide NPs leads to their loss from circulation and eventual accumulation in the bladder within as little as 30 mins of injection into a human or animal recipient (Sherwood et al., (2017) Nanoscale 9: 11785). The interwoven iron oxide NPs of the compositions of the disclosure, wherein the iron oxide NPs are attached to or embedded within TA/PVPON bilayers, however, provide capsules not only with MR imaging functionality but also allow for synergistic functional enhancements for the capsules to act as theranostic systems. For example, embedding ultra-small iron oxide NPs into TA/PVPON bilayer microcapsules provides T₁ and T₂ MRI contrast equal to that of gadolinium but at a fraction of the concentration of the agent, while also increasing the sensitivity of the capsule shell to ultrasound.

The soft multilayer microcapsules of the disclosure used as a drug delivery platform is advantageous since rigid delivery vehicles suffer from rapid blood clearance (Neuberger et al., (2005) J. Magn. Magn. Mater. 293: 483), tissue toxicity (Rose: Jr & Choi (2015) Am. J. Med. 128: 943), and activation of complex immune response. Soft polymeric drug vehicles can also preferentially accumulate in tumors because of their enhanced permeability and retention arising from leaky cancerous vasculature (Akimoto et al., (2014) J. Controlled Release 193: 2; Adair et al., (2010) ACS Nano 4: 4967; Larson & Ghandehari (2012) Chem. Mater. 24: 840). Remarkably, the upper size limit for cell internalization of soft particulates by non-phagocytotic cells is much higher than that of rigid inorganic or polymeric NPs that are excluded from cellular uptake at sizes greater than 150-200 nm; soft and flexible particulates ranging even from 3-5 μm have been shown to be internalized by cells (Xue et al., (2015) ACS Appl. Mater. Interfaces 7: 13633; Shimoni et al., (2013) ACS Nano 7: 522; Kozlovskaya et al., (2014) ACS Nano 8: 5725; Alexander et al., (2015) Adv. Health. Mater. 4: 2657; Gratton et al., (2008) Proc. Natl. Acad. Sci. U.S.A. 105: 11613).

While the shell of LbL-assembled H-bonded capsules exists as a nanothin network surrounding the encapsulated payload (such as agent), it is amenable to the formation of increased porosity upon mechanical disruption. The primary mechanism of capsule shell rupture is mechanical force that causes mechanical damage via inertial cavitation (de Jong et al., (1991) Ultrasonics 29: 324; Sirsi et al., (2014) Adv. Drug Delivery Rev. 72: 3). In the case of the (TA/PVPON/Fe₂O₃) capsule compositions of the disclosure, the incorporated ultra-small Fe₂O₃ NPs increase the susceptibility of the drug delivery constructs to ultrasound-induced oscillation by increasing the material density of the capsule shell (Skirtach et al., (2007) J. Mater. Chem. 17: 1050). A similar change in capsule shell permeability was reported for Co@Au NP-modified (PSS/PDDA) microcapsules to which an oscillating magnetic field was applied (Lu et al., 2005) Langmuir 21: 2042). In the case of these polyelectrolyte capsules, however, the oscillation of the shell due to the magnetic NPs was observed to create temporary, switchable porosity and allow influx of FITC-labeled dextrans.

For the NP-modified H-bonded shells of the present disclosure, oscillation induced by applied ultrasound likely causes rearrangement of the labile H-bonded shell architecture and opened co-requisite temporary pores in the shell. This is also in agreement with reports of PSS/PAH capsules with iron oxide NPs embedded into the polymer shell [Fe₃O₄/(PSS/PAH)₈] that broke into pieces after 60 sec sonication at 377 Wcm⁻², while particle-free (PSS/PAH)₈ capsules only deformed under the same treatment (Shchukin et al., (2006) Langmuir 22: 7400). However, the power intensity used in the study with the Fe₂O₃-TA/PVPON bilayer constructs of the disclosure was far below that mark (not exceeding 14 Wcm⁻² during the high intensity ultrasound treatment), even though therapeutic high intensity ultrasound may go well beyond 100 Wcm⁻² (Kiessling et al., (2014) Adv. Drug Delivery Rev. 72: 15; Miller et al., (2012) J. Ultrasound in Med. 31: 623).

The controlled release of DOX by capsules comprising the TA/PVPON/Fe₂O₃ Layer-by layer structures of the disclosure in response to ultrasound is an important significant feature advantageous for their use as applied drug delivery agents. In addition to mediating the capsule shell permeability, ultrasound plays a role in the actual delivery and uptake of the drug. In actual blood flow, the sonoporation effect, in which ultrasound energy enhances the permeability of cellular membranes, can help sequester the released drug into cells (Melodelima et al., (2004) Ultrasound Med. Biol. 30: 103; Huynh et al., (2015) Nat. Nanotechnol. 10: 325). Furthermore, tumor microvasculature has fenestrations ranging from 300 nm to 1.2 μm, depending upon the microenvironment and the tumor type (Hobbs et al., (1998) Proc. Natl. Acad. Sci. U.S.A.: 95: 4607). These fenestrations, with vascular permeability and hydraulic conductivity significantly higher than in normal tissues (Jain (1988) Cancer Res. 48: 2641), serve as a basis for the enhanced permeation and retention (EPR) effect (Fang et al., (2011) Adv. Drug Delivery Rev. 63: 136).

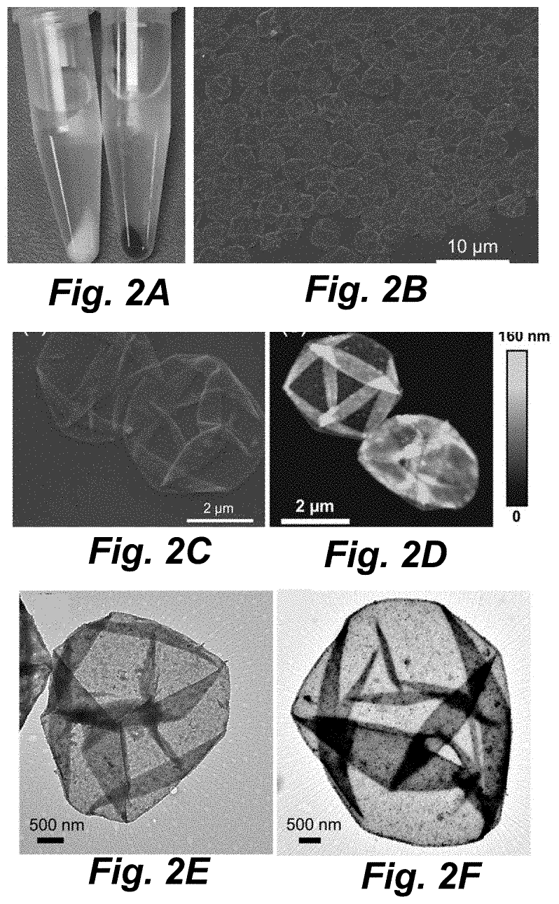

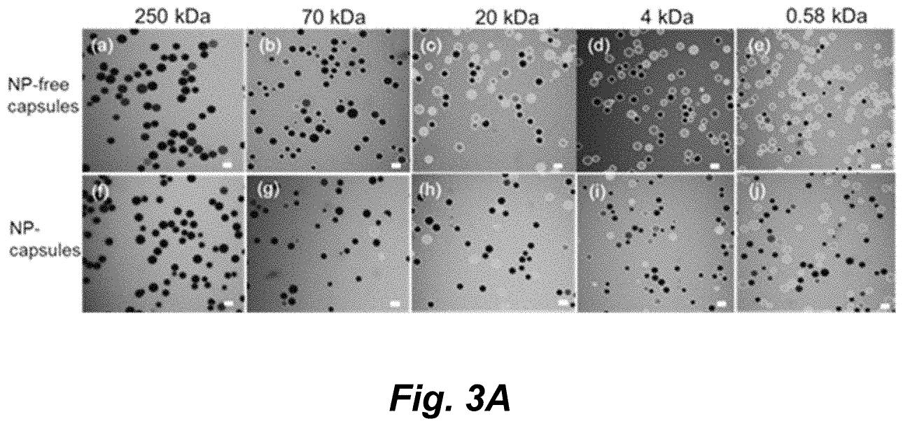

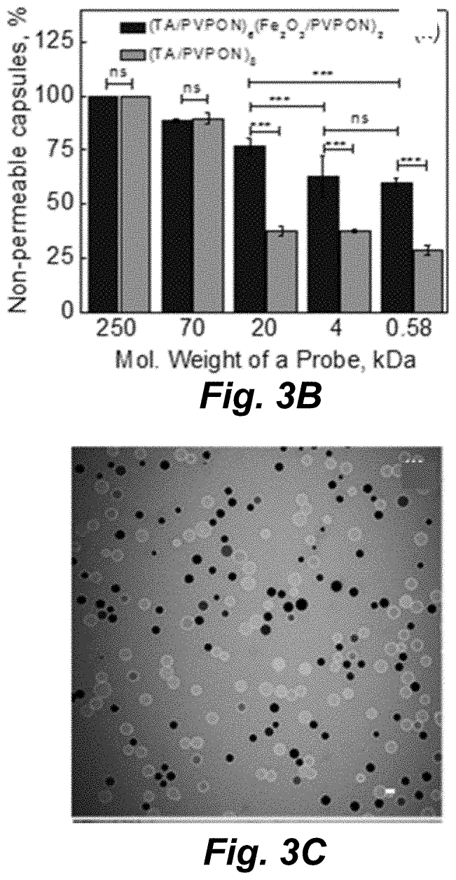

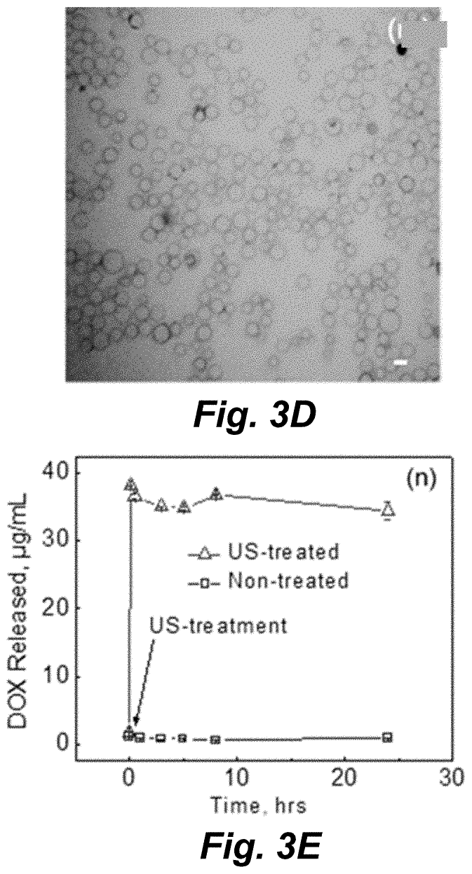

An advantage of the LBL structure of the capsules of the disclosure is that the iron oxide NPs can be included into the capsule shell in a layer-wise manner due to hydrogen-bonded interactions between tannic acid ligands on the particle surfaces and PVPON. The base capsule shell architecture of (TA/PVPON)₆ was compared with the NP-decorated architecture of (TA/PVPON)₆(Fe₂O₃/PVPON)₂ to demonstrate the top layer effect, as shown in the photographs, AFM, and TEM images of FIGS. 2A-2E. Also, the effect of the NPs on the shell permeability was determined by comparing (TA/PVPON)₆(Fe₂O₃/PVPON)₂ capsules to those with a (TA/PVPON)₈ architecture (having matched the total number of bilayers), as shown in FIGS. 3A, 3C, and 3D.

It was found that two Fe₂O₃ NP layers were sufficient to maximize MR imaging contrast while maintaining a shell flexibility and permeability appropriate for drug release, but this amount can be tailored to match the needs of the application if different drugs were encapsulated or different contrast intensity was required. Additionally, the use of PVPON as an outermost layer proved advantageous in increasing the capsule circulation by preventing accumulation of proteins that would inhibit the delivery of drugs and flag the delivery agents for rapid clearance.

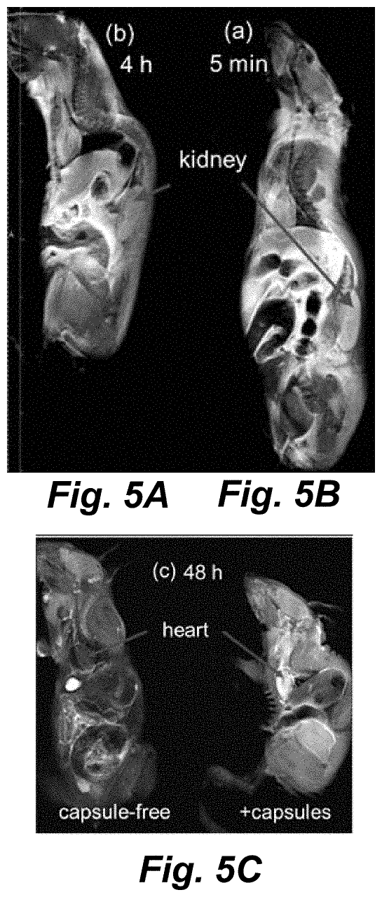

Bare 4-nm iron oxide NPs coated with TA showed T₁-weighted MRI contrast on a 9.4 T Bruker pre-clinical MRI scanner but were quickly cleared by the kidney and ended up in the bladder after 30 min of administration (Sherwood et al., (2017) Nanoscale 9: 11785). Drug-loaded capsules can travel to relevant biological locations to be used as theranostic agents and, as shown in the in vivo MR imaging data of FIG. 5 the capsules of the disclosure can circulate continuously in the blood stream for at least 48 h, an improvement in circulation compared to non-encapsulated Fe₂O₃ NPs.

The demonstrated ability of the capsules of the disclosure to promote brightness in T₁-weighted imaging is particularly interesting as the capsules are 3 μm in diameter in comparison to T₁ contrast agents known in the art or constructs that are nm-sized and tend to become better T₂ agents as size increases (Sandiford et al., (2013) ACS Nano 7: 500; Kim et al., (2011) J. Am. Chem. Soc. 133: 12624; Weissleder et al., (1990) Radiology 175: 489). A particle is now developed that is characterized with the circulation behaviors of a 3 μm object but has the T₁ contrast enhancement behavior of molecular and nm-sized agents. As demonstrated previously, polymeric capsules can deform mechanically to fit into spaces smaller than their diameter (Chen et al., (2017) ACS Nano 11: 3135; Sun et al., (2015) Chem. Sci. 6: 3505). This is useful in diagnostic imaging as the softness of the (TA/PVPON) capsules facilitates reversible fluid-like deformation in a similar manner to red blood cells; it has been shown earlier that 2 μm-sized (TA/PVPON) capsules can extravasate through 0.8 μm membrane pores under 18 psi (Alexander et al., (2015) Adv. Health. Mater. 4: 2657).

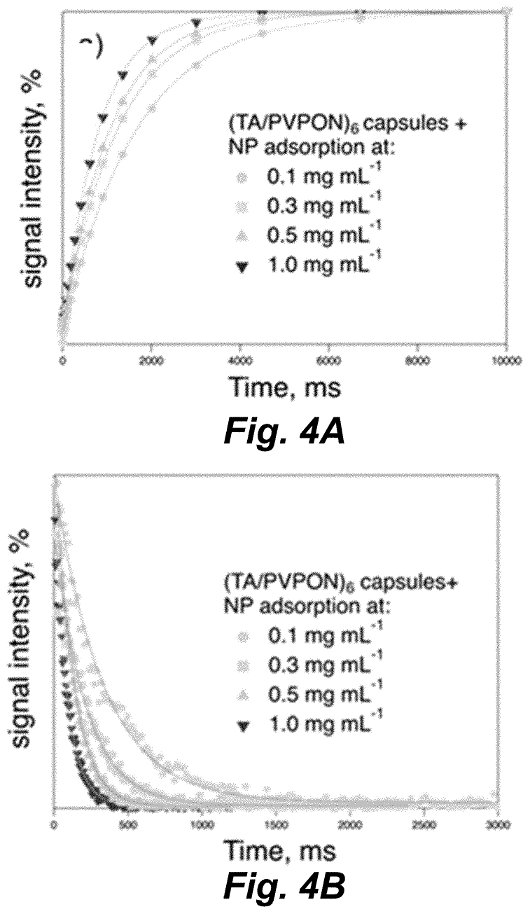

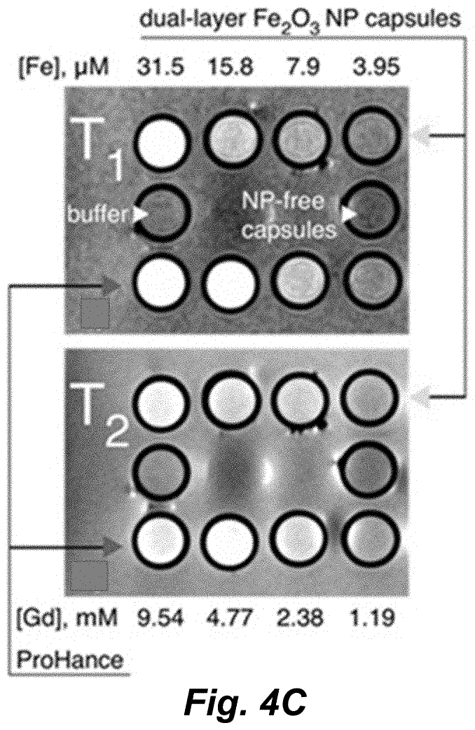

In addition to the advantageous T₁ contrast predicted by relaxometry, the (TA/PVPON/Fe₂O₃) hybrid capsules of the disclosure displayed contrast enhancement in T₂-weighted images, as shown in FIG. 4C, bottom panel, this increased brightness for the capsule suspensions being useful in tracking the capsules in vivo. This is a significant advantage since T₂-weighted images are often obtained in standard clinical MRI scans and can highlight abnormal levels of water diffusion in certain tissues. Additionally, an approximately 8% change in T₂ contrast was observed (FIG. 10) in response to applied ultrasound, which can be useful to confirm delivery of the encapsulated drug.

Statistical analysis (unpaired, two-tailed t-tests) of the contrast change given the pixel counts, mean, and SD gave a P value <0.0001, which denotes a result of high significance. The contrast change likely occurred due to the change in proximity of the NPs within the shell as the shell oscillates and allowed polymer rearrangement under the applied ultrasound. A similar effect was shown for magnetite NPs in polyelectrolyte PSS/PAH capsules in which the distance between NPs in the shell layers was shown to significantly affect the contrast intensity in both modes (German et al., (2016) Phys. Chem. Chem. Phys. 18: 32238). Since T₂ effects are more influenced by magnetic susceptibility, this can explain the change in T₂ contrast for with the capsule compositions of the disclosure in which the NPs themselves are not ferromagnetic and therefore do not see the same effect in T₁-weighted images.

Ultrasound-triggered release of encapsulated DOX was shown to increase the concentration of drug in tumors while also preventing major localization in off-target organs. While the long-term effects of delivered DOX in the tumors were not determined, it was demonstrated that the increased DOX delivery was not attributable to differences in capsule concentration between the ultrasound-treated and untreated control tumors as relaxometric iron quantification on immediately excised tumors revealed that no statistical difference in concentration of iron could be found between the two tumors. Using nanothin (TA/PVPON) polymer capsules with iron oxide NPs pharmacologically active agents, therefore, can be encapsulated and safely triggered to release relevant payloads using focused ultrasound. The presence of the iron oxide NPs permitted MR localization of drug containing capsules in circulation. This approach can deliver localized higher concentrations of the payload targeted at the tumor site while reducing off-target sequestering and toxicity.

Provided are hybrid (TA/PVPON/Fe₂O₃) capsules with excellent biocompatibility, long circulation, and MRI contrast in both T₁ and T₂ imaging modes, a method for their assembly, and methods for their use in imaging and targeting delivery of pharmacologically active agents. The (TA/PVPON)₆(Fe₂O₃/PVPON)₂ capsules of the disclosure were shown to have similar MR imaging contrast to a commercial agent ProHance (gadoteridol) at only 0.3% of the molar concentration of the metal. Accordingly, the capsules of the disclosure are advantageous for use as contrast enhanced imaging agents in vivo due to their circulation in the blood for at least 48 h as evidenced by MRI in a mouse model of breast cancer. A mild 14 Wcm⁻² unfocused ultrasound treatment was sufficient to release 35 μg mL⁻¹ of DOX from the Fe₂O₃ nanoparticle-modified 8-bilyaer H-bonded capsule, while it is known in the art that ultrasound power intensities greater than 350 Wcm⁻² were needed to break open NP-modified 8-bilayer polyelectrolyte [Fe₃O₄(PSS/PAH)] capsules (Shchukin et al., (2006) Langmuir 22: 7400). In addition, HIFU application to targeted tumors was shown to be sufficient to release anti-cancer therapeutics locally; a 16-fold higher concentration of Doxorubicin was measured in the target tumors compared to off-target organs including the spleen, liver, kidney, and lung.

The in vivo results obtained with the compositions of the disclosure also provide evidence that MRI-guided ultrasound-triggered drug delivery, as a non-invasive method, is advantageous for higher treatment precision as a result of real-time guidance by MR. Indeed, it was found that the T₂ contrast intensity of a capsule suspension of the disclosure changed by 8% after application of unfocused ultrasound at a low power intensity of only 14 Wcm⁻², which can be useful in confirming the manipulation of the capsule shell by ultrasound irradiation. The encapsulation, release, and imaging strategy provided by this approach enables the use of MRI guidance for targeted drug delivery while potentially improving treatment efficacy. Accordingly, the compositions of the disclosure provide a delivery system that can deliver to a target a pharmacologically active agent, or agents, that has been encapsulated by the TA/PVPON/Fe₂O₃ LBL capsules.

One aspect of the present disclosure, therefore, encompasses embodiments of a composition comprising a layer-by-layer plurality of polymer bilayers, wherein each polymer bilayer can comprise a polymer layer hydrogen-bonded to a polyphenolic tannin layer, and wherein at least one of the bilayers can further comprise a plurality of iron oxide nanoparticles attached thereto.

In some embodiments of this aspect of the disclosure, the composition can comprise from 1 to about 20 polymer bilayers.

In some embodiments of this aspect of the disclosure, the composition can comprise 6 polymer bilayers.

In some embodiments of this aspect of the disclosure, the polymer layer of each bilayer can be a poly(N-vinylpyrrolidone) layer.

In some embodiments of this aspect of the disclosure, the iron oxide nanoparticles can comprise ferric oxide and tannic acid.

In some embodiments of this aspect of the disclosure, the plurality of iron oxide nanoparticles can be attached to at least one polymer layer.

In some embodiments of this aspect of the disclosure, the at least one polymer layer having the iron oxide nanoparticles attached thereto can be a poly(N-vinylpyrrolidone) layer.

In some embodiments of this aspect of the disclosure, the composition can be a capsule defining an internal volume.

In some embodiments of this aspect of the disclosure, the layer-by-layer composition is deposited as a capsule encapsulating a solid core substrate.

In some embodiments of this aspect of the disclosure, the core substrate is in contact with a polyphenolic tannic acid layer of a bilayer.

In some embodiments of this aspect of the disclosure, the composition can further comprise a plurality of poly(N-vinylpyrrolidone) layers, each of said poly(N-vinylpyrrolidone) layers alternating with a layer of iron oxide-tannic acid nanoparticles.

In some embodiments of this aspect of the disclosure, the composition can further comprise an outer poly(N-vinylpyrrolidone) layer encapsulating the layer-by-layer composition.

In some embodiments of this aspect of the disclosure, the outer poly(N-vinylpyrrolidone) layer encapsulating the layer-by-layer composition can comprise a functional moiety attached thereto.

In some embodiments of this aspect of the disclosure, the outer poly(N-vinylpyrrolidone) layer encapsulating the layer-by-layer composition can comprise a functional moiety attached thereto, the functional moiety being selected from the group consisting of: a detectable moiety, an immunomodulatory molecule, a growth factor, a cell receptor ligand, a polypeptide cell receptor, or any combination thereof.

In some embodiments of this aspect of the disclosure, the core substrate can comprise at least one pharmacologically active agent.

In some embodiments of this aspect of the disclosure, the composition can encapsulate at least one pharmacologically active agent within the internal volume.

In some embodiments of this aspect of the disclosure, the core substrate can be removable.

Another aspect of the disclosure encompasses embodiments of a capsule, wherein the capsule can comprise a wall encapsulating a pharmacologically active agent, wherein the wall of the capsule can comprise: a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer; a plurality of poly(N-vinylpyrrolidone) layers, each of said poly(N-vinylpyrrolidone) layers alternating with a layer of iron oxide-tannic acid nanoparticles; and an outer poly(N-vinylpyrrolidone) layer.

In some embodiments of this aspect of the disclosure, the outer poly(N-vinylpyrrolidone) layer can comprises a functional moiety attached thereto.

In some embodiments of this aspect of the disclosure, the outer poly(N-vinylpyrrolidone) layer can comprises a functional moiety attached thereto, the functional moiety being selected from the group consisting of: a detectable moiety, an immunomodulatory molecule, a growth factor, a cell receptor ligand, a polypeptide cell receptor, or any combination thereof.

In some embodiments of this aspect of the disclosure, the capsule is mixed with a pharmaceutically acceptable carrier.

Still another aspect of the disclosure encompasses embodiments of a method of generating a layer-by layer composition, wherein said layer-by layer composition comprises an MRI contrast agent and a pharmacologically active composition, the method comprising the steps of: (a) obtaining a core substrate particle comprising a pharmacologically active agent; (b) obtaining a population of tannic acid-modified iron-oxide nanoparticles; (c) contacting the porous silica core of step (a) with a solution of a cationic polymer, thereby coating the porous silica core particle with the cationic polymer; (d) encapsulating the porous silica core particle of step (c) by depositing thereon a capsule comprising a layer-by-layer polymer coating, wherein said polymer coating comprises a plurality of tannic acid-poly(N-vinylpyrrolidone)-bilayers, wherein the tannic acid layer of a first bilayer is in contact with the porous silica core; (e) depositing a plurality of tannic acid-modified iron-oxide nanoparticles on a poly(N-vinylpyrrolidone) layer of a bilayer; (f) depositing a plurality of alternating poly(N-vinylpyrrolidone)-tannic acid-modified iron-oxide nanoparticle layers on the surface of the product of step (e); (g) depositing an outer poly(N-vinylpyrrolidone) layer on the surface of the product of step (f); and (h) removing the silica core from the capsule while leaving the pharmacologically active agent within the capsule.

In some embodiments of this aspect of the disclosure, the method can further comprise the step of attaching a functional moiety to the outer poly(N-vinylpyrrolidone) layer, the functional moiety being selected from the group consisting of: a detectable moiety, an immunomodulatory molecule, a growth factor, a cell receptor ligand, a polypeptide cell receptor, or any combination thereof.

Yet another aspect of the disclosure encompasses embodiments of a method of delivering a pharmacologically active agent to a patient in need thereof, the method comprising the steps: (a) administering to a patient a pharmacologically active composition comprising a capsule, wherein the capsule comprises a wall encapsulating a pharmacologically active agent, wherein the wall of the capsule comprises: a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer; a plurality of poly(N-vinylpyrrolidone) layers, each of said poly(N-vinylpyrrolidone) layers alternating with a layer of iron oxide-tannic acid nanoparticles; and an outer poly(N-vinylpyrrolidone) layer; (b) monitoring by magnetic resonance imaging (MRI) the delivery of the pharmacologically active composition to a selected site within the patient; and (c) administering an ultrasound emission to the patient, wherein the ultrasound emission has a frequency and intensity that disrupts the wall of the capsule of the pharmacologically active composition within the patient, thereby releasing the pharmacologically active agent to a tissue of the selected site patient.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

General: Poly(N-vinylpyrrolidone) (PVPON), tannic acid (TA), phosphate salts, and hydrofluoric acid were purchased from Fisher scientific and used as delivered. Porous silica cores were purchased from Restek and YMC. Chemicals for nanoparticle synthesis and surface functionalization: all of the chemical reagents were purchased and used without further purification. (FeCl₃, ACROS, 98%), sodium oleate (NaOA, TCL, 95%), oleic acid (OA, Fisher, 95%), oleyl alcohol (OL, Alfa Aesar, 80-85%), trioctylphosphine oxide (TOPO, Sigma-Aldrich, 90%), 1-octadecene (Sigma-Aldrich, 90%), chloroform (Sigma-Aldrich, 99 acetone (BDH, 99.5%), hexane (BDH, 100%), ethanol (Amresco, 100%), (4-(2-hydroxyethyl)-1-piperazineethanesulfonic acid (HEPES) buffer (OmniPur), and tannic acid (Acros, 95%). Doxorubicin hydrochloride was purchased from LC laboratories. Ultrapure deionized water (0.055 μS/cm) was used for solution preparation (Siemens). A Bruker minispect NMR was used for relaxometry (mq60, 1.4 T, 60 MHz). MRI was performed on a Siemens Allegra 3 T MRI and a Bruker 9.4 T animal scanner. In-situ ultrasound treatments were conducted using either a Fisher sonic dismembrator with a 3 mm diameter tip or a custom modular ultrasound generating system (Chen et al., (2017) ACS Nano 11: 3135) (E&I RF amplifier, Techtronix function generator, and Olympus ultrasonic transducers).

### Example 2

Synthesis of iron oxide nanoparticles: The ultrasmall iron oxide NPs 4 nm in diameter were synthesized and surface functionalized as previously described (Sherwood et al., (2017) AIP Advances 7: 056728). Briefly, the NPs were synthesized by decomposing an iron oleate complex in diphenyl ether at 250° C. During the process, oleic acid and trioctylphosphine oxide (TOPO) was added as a surface capping molecule, and oleyl alcohol was used as a reducing agent. After a two-minute reaction at 250° C., the reaction mixture was rapidly cooled down to room temperature and the NPs were collected by centrifugation (15,000 rpm; 2 min). After rinsing with ethanol, the NPs were dried under vacuum overnight. The well-dried NPs were re-dispersed in hexane to obtain a stock solution of 5 mg mL⁻¹ for the ligand exchange process. Subsequently, the hydrophobic coating of the NPs was replaced with TA following the established protocols (Sherwood et al., (2017) Nanoscale 9: 11785). Briefly, 1 mL of nanoparticle stock solution was mixed with 2 mL of TA solution (HEPES, pH=7, 12 mg mL⁻¹), and then sonicated in an ice bath for 5 minutes using a tip sonicator (25% amplitude, pulse 3 s-on and 1 s-off) to form an emulsion. Then, the emulsion was mixed with equal volume of acetone to facilitate ligand exchange process. Finally, the NPs were centrifuged out of solution (15,000 rpm, 15 min) and rinsed three times with DI water followed by nanoparticle dispersing in 10 mM HEPES buffer and used for synthesis of composite multilayer capsules.

### Example 3

Synthesis of (TA/PVPON)ₙ and (TA/PVPON)ₙ/(Fe₂O₃/PVPON)ₙ multilayer capsules: (TA/PVPON) capsules were prepared by coating TA and PVPON layers sequentially on the sacrificial cores in 0.01 M pH=6 phosphate buffer. Specifically, 40 mg of porous (3 μm) silica particles were added to a 1.5 mL Eppendorf centrifuge tube. A 1 mg mL⁻¹ aqueous PEI solution was first adsorbed on the particles for 10 minutes during vigorous shaking (2000 rpm). The particle solution was centrifuged at 8000 rpm for 30 s and the supernatant was removed. The particles were then rinsed 3 times with 0.01 M pH=6 phosphate buffer. Tannic acid (TA) (0.5 mg/mL, 0.01 M phosphate buffer, pH=6) was allowed to adsorb onto particle surfaces for 10 minutes during vigorous shaking. After centrifuging and rinsing with phosphate buffer, the particles were exposed to PVPON solution (0.5 mg mL⁻¹, 0.01 M phosphate buffer, pH=6) for 10 minutes during shaking (2000 rpm). The suspension was centrifuged and rinsed as with the buffer as in the previous step. Alternating exposure of the particles to the polymer solutions was continued until the desired number of (TA/PVPON) bilayers (n) was achieved. For example, but not intended to be limiting, n can be in the range from 1 to about 20, from 1 to about 10, from 1 to about 10, from 1 to about 8, from 1 to about 6, from 1 to about 4, and from 1 to about 2. In all embodiments of the multi-bilayer constructs of the disclosure the construct may further have a biocompatible PVPON layer as the layer most distal from the first formed bilayer. This PVPON layer may be further modified by the attachment, either non-covalently or covalently, of at least one compound or moiety that can modulate the biological properties of the construct or is effect when administered to a recipient animal or human.

To obtain hollow (TA/PVPON)ₙ microcapsules, the sacrificial silica cores were dissolved using 8% hydrofluoric acid (HF) for 3 days followed by dialysis against 0.01 M phosphate buffer at pH=7.4. To embed Fe₂O₃ NPs within the capsule multilayer shell, the (TA/PVPON)ₙ capsules solutions were diluted to a fixed concentration of approximately 10⁸ capsules mL⁻¹ (counted using a hemacytometer), exposed to a 1 mg mL⁻¹ aqueous solution of the TA-coated Fe₂O₃ NPs and shaken for 12 h (2000 rpm). After that, the capsules were transferred into 1-mL Float-a-Lyzer tubes (SpectrumLabs, MWCO 20 kDa) and dialyzed exhaustively against a 0.01 M phosphate buffer at pH=7.4 for a week to separate (TA/PVPON)ₙ/Fe₂O₃ capsules from the free non-adsorbed NPs. To form capsules with a hydrophilic outer layer, PVPON was deposited on the capsules as the topmost layer (0.5 mg mL⁻¹, pH=6, 0.01 M phosphate buffer), and the capsules were rinsed 3 times with the corresponding phosphate buffer.

### Example 4

Scanning Electron Microscopy (SEM): SEM analysis of the (TA/PVPON)₆(Fe₂O₃/PVPON)₂ capsules was performed using a FEI Quanta™ FEG microscope at 10 keV. Samples were prepared by depositing a drop of a capsule suspension on a silicon wafer and allowing it to dry at room temperature. Before imaging, dried specimens were sputter-coated with 5 nm silver film using a Denton sputter-coater.

### Example 5

Atomic Force Microscopy (AFM): AFM height images of (TA/PVPON)₆(Fe₂O₃/PVPON)₂ capsules were collected on dry samples using Multimode 8 (Bruker) in the Soft Tapping mode in air. AFM probes were purchased from Bruker (resonance frequency ˜300 kHz, tip radii 10 nm). For the preparation of capsules for AFM imaging, a drop of the capsule suspension was placed on a cleaned silicon wafer and dried in air prior to AFM imaging. The capsule single wall thickness was determined as half of the height of the collapsed flat regions of dried capsules using Nanoscope software 1.5 (Bruker) for the analysis.

### Example 6

Transmission electron microscopy (TEM): TEM was performed on a Tecnai Spirit T12 electron microscope operated at 100 kV. To analyze the nanoparticle-containing capsules, the specimens were placed on a carbon-coated copper grid (Electron Microscopy Sciences, 200 mesh) and dried in air before TEM analysis.

### Example 7

Confocal Laser Scanning Electron Microscopy (CLSM): Confocal Images of the capsules were obtained with Nikon A1R+confocal microscope equipped with a 63×oil immersion objective. To observe capsule shape and investigate the capsule permeability toward small and large molecule fluorescent probes, a drop of a hollow capsule dispersion was added to 8-well Lab-Tek chambers (Electron Microscopy Sciences), and settled for 5 hours. Then 0.2 mL of fluorescein isothiocyanate (FITC)-labeled dextran (FITC-dextran) or Alexa Fluor 488 fluorescent dye (1 mg mL⁻¹) were added to the capsule solution in the CLSM chamber and left for 15 minutes before starting capsule imaging.

### Example 8

Capsule incubation in FBS: For the experiments with capsules suspended in FBS, 100% FBS was first allowed to melt at 37° C. 10⁸ capsules mL⁻¹ were pelleted by centrifugation in an Eppendorf tube and the supernatant was replaced with 100% FBS. The suspensions were incubated at 37° C. with intermittent vortexing over the course of 24 h. At 4 and 24 h one set of capsules was pelleted and the supernatant replaced with buffer before MR imaging next to capsules that had been incubated without the FBS.

### Example 9

High Pressure Liquid chromatography-Mass spectrometry (HPLC-MS): Mouse organ homogenates were prepared by dissolving the excised tissues in RIPA Lysis buffer (10 mM Tris-Cl (pH 8.0); 1 mM EDTA; 1% Triton X-100; 0.1% sodium deoxycholate; 0.1% SDS; 140 mM NaCl). A protease inhibitor (1 tablet, Thermo Scientific) was added to 7 mL of the RIPA Lysis buffer before lysate preparation. The homogenates were prepared for HPLC-MS by vortexing 100 μL of the homogenate with a known concentration of dipyridamole in 300 μL of 1:4 ethanol/acetonitrile. The solutions were filtered on a Captiva ND 0.2 μm protein precipitation plate and 5 μL was injected into the HPLC-MS (Atlantis T3 5 μm 4.6×50 mm column). A calibration curve was prepared in pH=7 phosphate buffer with liver homogenate; and the internal standard was used to correct for matrix ion suppression.

### Example 10

NMR relaxometry measurements: The method for determining iron concentrations using a Bruker minispec was used in accordance with a previous report (Sherwood et al., (2016) Nanoscale 8: 17506). In brief, a standard curve was created by plotting the relaxation rate (1/T₁ and 1/T₂) of FeCl₃ solutions at various iron concentrations (0.01, 0.02, 0.03, 0.05, 0.08, 0.1, 0.2, 0.3, 0.4, and 0.5 mM). The T₁ and T₂ relaxation times of these solutions were measured in three replicas in order to ensure the accuracy of the standard curve. From the standard curve, relaxivity of Fe³⁺ was obtained, which was subsequently used to estimate the iron concentration of the (TA/PVPON)₆(Fe₂O₃/PVPON)₂ capsule solutions. To quantify iron concentration in the capsules, (TA/PVPON)₆(Fe₂O₃/PVPON)₂ capsules were exposed to 5% (wt) nitric acid for 10 min to dissolve iron oxide NPs, and iron concentration in supernatants was quantified by NMR.

### Example 11

Atomic absorption spectroscopy: Iron standards were prepared by dissolving polished iron wire (2 mm×5 cm; Alfa Aesar) in 10:1 HCl/HNO₃ and diluting with deionized water to known concentrations in the range of 1-10 ppm. Standards were measured in triplicate using 1.5 s measurement times to construct a calibration curve with an R² of 0.996. Capsule suspensions were pelleted (5×10⁸ capsules mL⁻¹) by centrifugation at an RCF=4,000 for 8 min and the supernatant replaced with 10:1 v/v HCl/HNO₃ to digest the iron NPs (30 min). The solutions were filtered through 0.2 μm pore-size filters (Fisher Scientific) before being injected into the AAS instrument (Perkin Elmer AAS 3300 with Perkin Elmer Lumina Fe lamp). Each sample was measured in triplicate to determine Iron concentration based on the absorbance value.

### Example 12

Loading DOX in TA/PVPON Capsules: DOX hydrochloride (LC Laboratories) was converted to the free amine as described (Liu et al., (2014) Soft Matter 10: 9237). The DOX was then dissolved in CHCl₃ (12.5 mg mL⁻¹) and added to porous silica cores (40 mg) in a 1.5-mL Eppendorf tube using a 0.2-μm pore syringe filter. After sealing and shaking overnight on a Corning shaker, the centrifuge tube was opened and kept in a vacuum oven at 40° C. for 12 h. PEI was adsorbed onto the dried DOX-loaded cores from 1 mg mL⁻¹ aqueous solution for 10 min, and the cores were separated from the polymer solution by centrifugation. After a triple rinse with 0.01 M phosphate buffer at pH=6, the cores were coated with TA/PVPON multilayers as described previously for capsule synthesis. The silica cores were dissolved in 8% hydrofluoric acid to yield the DOX-(TA/PVPON)₆(Fe₂O₃/PVPON) capsules with encapsulated DOX. Concentration of capsules in solutions was determined using a hemocytometer. The DOX loading capacity of the capsules was determined as follows: after loading, multilayer deposition, and core removal, a suspension of capsules was diluted to 1×10⁸ capsules mL⁻¹ in 0.01 M phosphate buffer at pH 7.4 and treated with 20 KHz ultrasound at 100 Wcm⁻² for 180 s to completely destroy all capsules. The destruction of capsules was monitored by optical microscopy until no spherical particles remained in solution. The destroyed capsule solution was centrifuged for 10 min to pellet the amorphous complexes of TA and PVPON and leave solvated DOX in the supernatant. The supernatant was measured via UV-vis and the concentration of DOX was calculated based on a standard calibration curve of absorbance at 480 nm. Finally, the concentration of DOX per mL of capsule solution was divided by the initial concentration of DOX loading to determine the loading efficiency.

### Example 13

Animal Model Preparation: Athymic nude female mice aged 4 to 6 wk (Charles River Laboratories) were used. The human breast cancer cell line MDA-MB-231 was obtained from American Type Culture Collection (ATCC, Manassas Va.) and maintained in Dulbecco's modified Eagle's medium supplemented with 10% fetal bovine serum (HyClone, Logan, Utah). Cells were cultured in 75 cm² flasks. At approximately 80% confluency, cells were harvested by trypsinization, counted with a hemocytometer, and diluted to a final concentration of 1.0×10⁷ cells/mL. Mice (n=4) were inoculated with 1×10⁶ cells (100 μl) subcutaneously in each flank to generate bilateral tumors. The mice that received ultrasound treatments were housed in a facility with a 9.4 T scanner, while the mice that received the injections to observe the circulation behaviors were housed in a facility with a 3 T scanner. Since both studies required immediate imaging after specific time points, the mice were imaged on different scanners as appropriate. After ultrasound treatment and MR imaging, animals were sacrificed, tumors were excised, bisected and fluorescence imaged. Additional organs (lung, liver, kidney, and spleen) were also collected for HPLC-MS.

### Example 14

Capsule ultrasound treatment: For high intensity ultrasonic treatment of capsules solutions in-situ, a 20 kHz Fisher FB120 sonic dismembrator (0.3 cm probe diameter) with tunable power output was used. Specifically, the ultrasonic probe was placed into a 1.5 mL Eppendorf tube containing capsule solution (10⁸ capsules mL⁻¹) and the time and power amplitude were set on the probe controller. Treatments were applied in 3×20 s intervals with 20 s rest periods in between for a total treatment time of 60 s. A digital thermometer was used to test the temperature of the capsule solutions before and after ultrasound treatment to detect any change in temperature. The power intensity was calculated by the following equation:

$$Power intensity ( \frac{W}{cm 2} ) = \frac{output power ( W )}{ultrasound treated area ( transducer horn area )}$$

The release of DOX from the capsules was measured by pelleting the capsule suspension via centrifugation after treatment with ultrasound and measuring the supernatant with released DOX using UV-vis. The DOX concentration was calculated from a standard calibration curve at 480 nm. The supernatant was returned to the capsules which were shaken at 25° C. on a Corning shaker at 2000 rpm between measurements over the 24 h time course.

### Example 15

In vivo capsule MRI: C57BL/6 female mice weighing 20-25 g (Charles River Laboratories) were used. Capsules and commercial contrast agent ProHance were injected systemically via tail vein. Mice received injections of either; 0.2 mL kg⁻¹ of ProHance diluted in 10 mL kg⁻¹ of saline, 10 mL kg⁻¹ of (TA/PVPON)₆Fe₂O₃PVPON capsules loaded with DOX at 2×10⁸ capsules mL⁻¹, or 10 mL kg⁻¹ of saline (3 mice per group). Separate mice were allowed to rest for 5 min, 4 h, and 48 h after injection to allow circulation of the capsules or Definity agent. Subsequently, the mice were sacrificed and immediately imaged on a Siemens MAGNETOM Prisma singo MR D13 3 T MRI to “freeze” the circulation time points.

### Example 16

In vivo MRI-guided ultrasound triggered drug release: Athymic nude female mice with the age of 4 to 6 weeks (Charles River Laboratories) were obtained and housed in accordance with UAB Institutional Animal Care and Use Committee (IACUC) guidelines. 10 mL/kg of 2×10⁸ capsules mL⁻¹ DOX-loaded (TA/PVPON)₆(Fe₂O₃/PVPON)₂ capsules were intravenously injected and allowed to circulate into tumor tissue. Animals were MR-imaged at 9.4 T (using the parameters stated above) before and after injection and ultrasound treatment. For tumor targeting, animals were anaesthetized (2-3% isoflurane), hair was removed from the target tumor surface and using an XYZ positioning system (Valmex, Bloomfield, N.Y.) the focused ultrasound probe (1 MHz, 0.50 in. Element Diameter, Standard Case Style, Straight UHF Connector, F=0.80 IN PTF; Olympus America Inc) was positioned with the focal point at the middle of the tumor mass. A water bath was coupled to the tumor surface with ultrasound gel. The ultrasound probe was lowered into the water bath at its target position. Then the animal was slowly infused with 30 μL kg⁻¹ of Definity, while ultrasound was applied to the target tumor. The non-targeted tumor was used as a control. Immediately, the animals underwent MR imaging: T₁-weighted MR images (10 coronal slices, 0.5×0.5×2 mm voxels, 1 mm gap, FOV=10×3.5×3 cm) of the animals were recorded pre-injection and every 30 min for 2 h post-injection on a Bruker BioSpec scanner (Bruker BioSpin, Billerica, Mass.) with a custom volume coil. A pneumatic pillow sensor placed under the mouse chest and connected through an ERT Control/Gating Module (SA Instruments) was used to acquire the mouse's respiratory cycle. The MRI sequence was actively gated to avoid acquisition during inhalation and exhalation. Animals were then sacrificed and tissues were collected. Fluorescence images on resected tumors were collected immediately with an IVIS Lumina. After IVIS imaging, tumors were split in half and flash frozen and for fluorescent microscopy and tissue processing. Unpaired t-tests were used to examine the statistical significance (P-value) between the MR and fluorescence ROI areas to determine capsule MRI contrast and DOX release. For MRI contrast enhancement analysis, following mean intensity projection of the tumor mass to form a 2D image, an ROI was manually drawn around the tumor in the MR image. This was done separately for each mouse, as there are slight differences in mouse positioning and tumor size. Total MR signal intensity within that ROI was measured and normalized by total pixel counts to quantify mean tumor intensity.

### Example 17

### Statistical Analysis

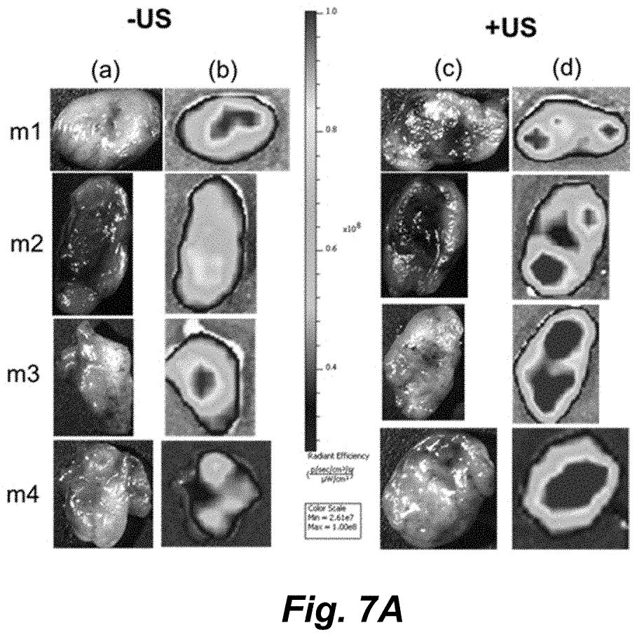

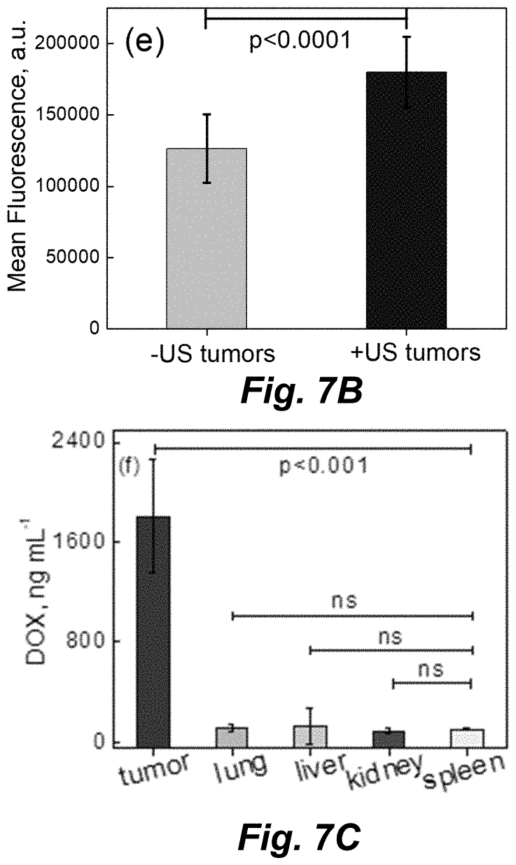

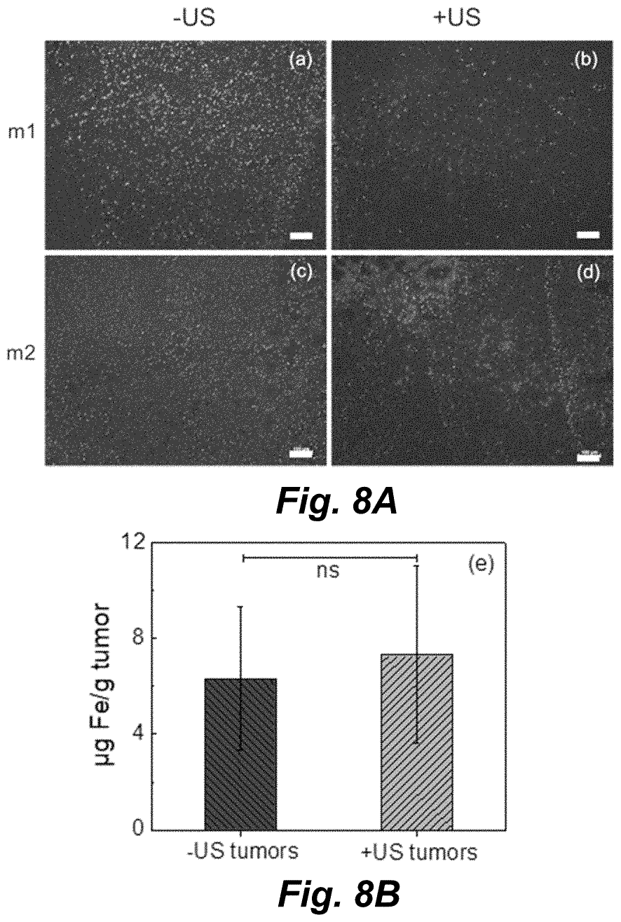

Pre-processing: The relaxivity data in FIGS. 4A-4C were normalized to the horizontal asymptotes corresponding to 90% for longitudinal magnetization and 0% for transverse magnetization before presentation. Mean fluorescence in FIG. 7B was calculated by dividing total counts by pixel area of the ROI drawn on each tumor in the microscopy software for the IVIS Lumina. The DOX concentrations obtained from HPLC-MS in FIG. 7C were corrected for matrix ion suppression using internal standards as discussed in the SI. The iron concentrations in FIG. 8B were normalized per g of tumor mass to account for differences in the mass of dissolved tissue for each of the four mice. Data in all other figures are presented directly in the units captured from measurement without normalization.

Presentation: All plots present data as mean±SD where markers or bars represent the numerical mean with vertical lines representing the standard deviation.

Sample size: The sample size (n) for each presented data set is given as follows and is stated in the corresponding figure captions as appropriate. Iron concentration in capsules via AAS and relaxometry: n=3 with 3 aliquots measured in each measurement. Capsule permeability in FIG. 3B: n=150 for each measurement of permeability with 50 capsules counted in 3 areas from the CLSM images. DOX release in FIG. 3E: n=3 with 3 aliquots taken from the capsule suspension for UV-vis measurement. Mean fluorescence in FIG. 7B: n=6313 and 7255 for total pixel counts of the untreated and ultrasound-treated tumors, respectively. DOX concentration in FIG. 7C: n=4 for each measurement with one set of harvested tissues for each of 4 mice. Iron concentration in FIG. 8B: n=4 for each measurement with 4 untreated and 4 ultrasound-treated tumors dissolved for relaxometry analysis.

Statistical methods: Unpaired, two-tailed T-tests were used to assess the statistical significance (P-value) of each data set by inputting the mean, SD and n values for each group. P-values are given directly on each applicable plot and “ns” is shown if no statistically significant difference was found. GraphPad software was used for all statistical analyses.

### Example 18

Assembly of capsule-based contrast agents: Multilayer (TA/PVPON)ₙ microcapsules were obtained using a hydrogen-bonded layer-by-layer (LbL) approach (Kozlovskaya (2015) Adv. Healthcare. Mater. 4: 686; Chen et al., (2013) Biomacromolecules 14: 3830; Liu et al., (2014) Soft Matter 10: 9237; Chen et al., (2017) ACS Nano 11: 3135), where the subscript n denotes the number of (TA/PVPON) bilayers. To produce (TA/PVPON)₆ microcapsules, TA and PVPON layers were deposited in alternating fashion onto porous 3 μm silica spheres from 0.5 mg mL⁻¹ polymer solutions at pH=6 followed by core dissolution in aqueous hydrofluoric acid (HF).

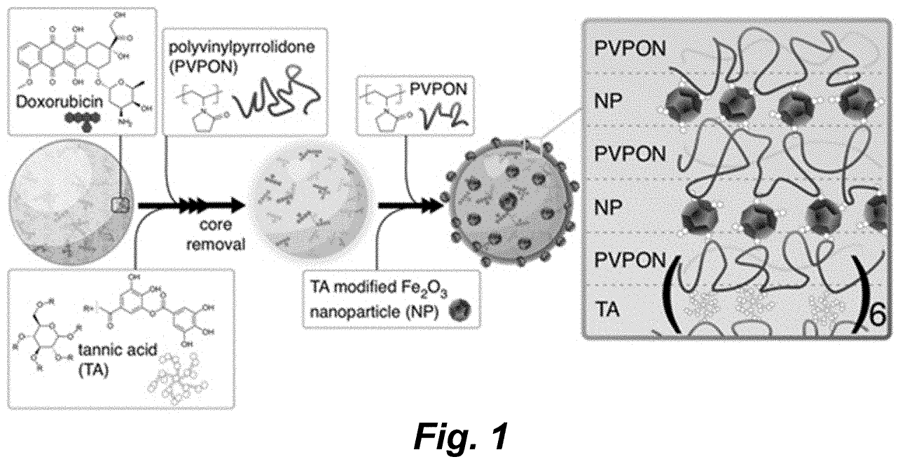

To imbue the capsules with MRI visibility, a layer of TA-modified Fe₂O₃ NPs was adsorbed onto the (TA/PVPON)₆ capsules followed by PVPON and a second Fe₂O₃/PVPON bilayer for a final shell architecture of (TA/PVPON)₆(Fe₂O₃/PVPON)₂ (as schematically shown in FIG. 1). The choice of TA as a NP surface ligand was made based on its ability to form multiple intermolecular hydrogen bonds with PVPON (Chen et al., (2013) Biomacromolecules 14: 3830; Liu et al., (2014) Soft Matter 10: 9237) and the PVPON capping was chosen to prevent capsule coagulation and cell recognition in vivo; in a similar manner to poly(ethylene glycol), PVPON has been shown to prevent protein adsorption on surfaces due to its highly hydrophilic nature (Andersen et al., (2011) Biomaterials 32: 4481; Gaucher et al., (2009) Biomacromolecules 10: 408).

After embedding Fe₂O₃ NPs in the capsule shell, the (TA/PVPON)₆(Fe₂O₃/PVPON)₂ capsules were found to be distinctively different from their NP-free templates with the capsule pellets becoming visibly darker in comparison (FIG. 2A). SEM analysis of the (TA/PVPON)₆(Fe₂O₃/PVPON)₂ capsules revealed complete core dissolution and that the capsules preserved their integrity after embedding of two layers of the nanoparticles (FIGS. 2B and 2C). AFM analysis of the capsules did not show any nanoparticle clusters at the capsule surfaces (FIG. 2D) and revealed the single wall thickness of the capsules to be 21±2 nm.

The presence of the Fe₂O₃ NPs within the capsule shell was confirmed by TEM analysis (FIGS. 2E and 2F) where unstained TEM images of the (TA/PVPON)₆(Fe₂O₃/PVPON)₂ capsules revealed the capsule shell with nanoparticles homogeneously distributed throughout (dense areas especially noticeable on the capsule folds) (FIG. 2F), unlike the NP-free capsules where the lighter nanoparticle-free capsule folds can be observed (FIG. 2E).

### Example 19

Capsule shell permeability: Embedding Fe₂O₃ NPs into the capsule shell can, along with affecting the capsule rigidity (Skirtach et al., (2007) J. Mater. Chem. 17: 1050; Pavlov et al., (2011) Soft Matter 7: 4341), change the permeability of the nanothin shell toward large and small molecules. To quantify the permeability of the capsule wall, (TA/PVPON)₈ and (TA/PVPON)₆(Fe₂O₃PVPON)₂ capsules were incubated in solutions of FITC-dextrans with molecular weights ranging from 4,000 to 250,000 Da. Confocal laser microscopy (CLSM) analysis revealed that while both the (TA/PVPON)₈ and (TA/PVPON)₆(Fe₂O₃/PVPON)₂ capsules were similarly impermeable to FITC-dextrans of 250,000 and 70,000 Da, the permeability of the latter was 2- and 1.7-fold less towards FITC-dextrans of 20,000 and 4,000 Da than that for NP-free (TA/PVPON)₈ capsules (FIG. 3A (Panels (a)-(d), (f)-(i)). Likewise, incorporation of the Fe₂O₃ NPs within the capsule shell significantly decreased the permeability to the small molecule Alexa Fluor 488 fluorescent dye (MW=580 Da) (FIG. 3A (Panels (e) and (j)). The CLSM images showed that 60% of the (TA/PVPON)₆(Fe₂O₃PVPON)₂ capsules became impermeable to the dye, while only 28% of the non-modified (TA/PVPON)₈ capsules remained closed to the dye after 15 min of exposure at pH=7.4 (FIG. 3A (Panels (e), (j), and (k)).

Embedding Fe₂O₃ NPs impacted the response of the capsule shell to ultrasound. After exposing (TA/PVPON)₆(Fe₂O₃/PVPON)₂ capsules to low intensity ultrasound resembling that which is used in diagnostic imaging (2.25 MHz; 115 mWcm⁻²; 15 min), 100% of the capsules became open to 580 Da hydrophilic Alexa Fluor 488 fluorescent dye (FIGS. 3B, 3D). This demonstrates the response of (TA/PVPON)₆(Fe₂O₃PVPON)₂ capsules to low intensity diagnostic ultrasound compared to previously reported NP-free (TA/PVPON) capsules that showed only partial opening (57±5%) to diagnostic ultrasound under the same conditions (Chen et al., (2017) ACS Nano 11: 3135).

The ability of the capsules of the disclosure to release DOX upon ultrasound treatment was explored using higher power intensity such as that used in ultrasonic therapy (Orsi et al., (2010) Am. J. Roentgenol. 195: W245). Based on the concentration of the DOX solution used for loading the silica cores and the loading capacity of the capsules the loading efficiency was about 13%. (TA/PVPON)₆(Fe₂O₃/PVPON)₂ capsules loaded with 0.8 pg DOX per capsule (1×10⁸ capsules mL⁻¹) released 35 μg mL⁻¹ of DOX under relatively mild unfocused ultrasound (20 kHz, 14 Wcm⁻², 60 s in 20 s bursts with 20 s rests) (FIG. 3E), corresponding to approximately 44% cumulative release of the loaded DOX. This burst drug release was not accompanied by any detectable heat, according to a digital thermometer inserted in the solution before and after ultrasound treatment, in the sample solution upon ultrasound treatment.

The release shown here contrasts against previously reported liposomal systems in which heat was required to release the drug (Kim et al., (2016) Mol. Pharmaceutics 13: 1528) and other more stable liposomal formulations to which 30-60 min of ultrasound-induced heating may be required to release significant amounts of the loaded drug (Grüll & Langereis (2012) J. Controlled Release 161: 317; de Smet et al., (2011) J. Controlled Release 150: 102). The negligible decrease in DOX release following the ultrasound-triggered burst shown in FIG. 3E can be attributed to the noncovalent interactions between the released DOX and the outside surface of the capsule wall.

### Example 20

MR imaging response in situ: As quantified by atomic absorption spectroscopy, the inclusion of two layers of iron oxide NPs within the capsule shell resulted in 1.76 μg Fe mL⁻¹ in the (TA/PVPON)₆(Fe₂O₃/PVPON)₂ capsule suspension (10⁸ capsules mL⁻¹), or 31.5 μM iron concentration. This quantification was supported by relaxometric measurements against a standard Fe calibration curve which showed the concentration of iron in the same architecture capsules to be 1.98 μg Fe mL⁻¹. The highly-controllable LbL approach used to embed the NPs in the capsule shell further allowed for tailoring the iron concentration of any individual NP layer as can be seen from the relaxivity curves in FIGS. 4A and 4B. In this case, different concentrations of the NP solution were allowed to adsorb on the surface of the capsules for a fixed time period before measurement. Increasing the concentration of the NP solution from 0.1 mg mL⁻¹ through to 1.0 mg mL⁻¹ of the Fe₂O₃ NPs during deposition resulted in increased enhancement of both T₁ and T₂ relaxation behavior as can be seen by the trend in the relaxation curves (FIGS. 4A and 4B).

Compared to free NPs in solution, with the relaxation rates r₁ and r₂ of 3.81 and 4.59 mM⁻¹ s⁻¹ at 1.4 T, respectively (Sherwood et al., (2017) Nanoscale 9: 11785; Sherwood et al., (2017) AIP Advances 7: 056728), inclusion of two layers of NPs in the capsule shell resulted in an enhancement in relaxivity with a 2.1-fold increase in r₁ to 7.91 mM⁻¹ s⁻¹ and a 3.2-fold increase in r₂ to 14.69 mM⁻¹ s⁻¹ as calculated from the relaxation rates. In comparison, the common MRI contrast agent ProHance (gadoteridol) has been shown to exhibit an r₁ of 4.1 mM⁻¹ s⁻¹ and an r₂ of 5.0 mM⁻¹ s⁻¹ at 1.5 T (Rohrer et al., (2005) Invest. Radiol. 40: 715).

MR images of NP-containing and NP-free capsules placed alongside solutions of gadoteridol were obtained using a clinical 3 T MRI scanner (FIGS. 4C and 4D). The comparable imaging contrast in T₁ (FIG. 4C, top panel) and T₂ (FIG. 4C, bottom panel) weighted imaging modes from the capsule and gadoteridol solutions is shown within serial 0.5×dilutions of the 10⁸ capsules mL⁻¹ suspension from left to right (FIG. 4C, top and bottom panels); Fe and Gd concentrations listed above and below image). Neither NP-free (TA/PVPON)₆ capsules nor a capsule-free buffer solution showed MRI contrast in either T₁ or T₂ modes.

Inclusion of the iron oxide NPs within the capsule wall enabled MR imaging contrast at a similar intensity to gadoteridol (FIG. 4C, top and bottom panels) at only 0.3% of the metal concentration as imaged with a clinical 3 T MRI scanner. This result highlights particular interest as our capsules promote excellent T₁ contrast (brightness in T₁-weighted images) but are 3 μm in diameter. In comparison, for most T₁ contrast agents, sizes under 15 nm are essential for promoting T₁ effects while T₂ effects tend to dominate in particles exceeding this size (Sandiford eta l., (2013) ACS Nano 7: 500; Kim et al., (2011) J. Am. Chem. Soc. 133: 12624; Weissleder et al., (1990) Radiology 175: 489).

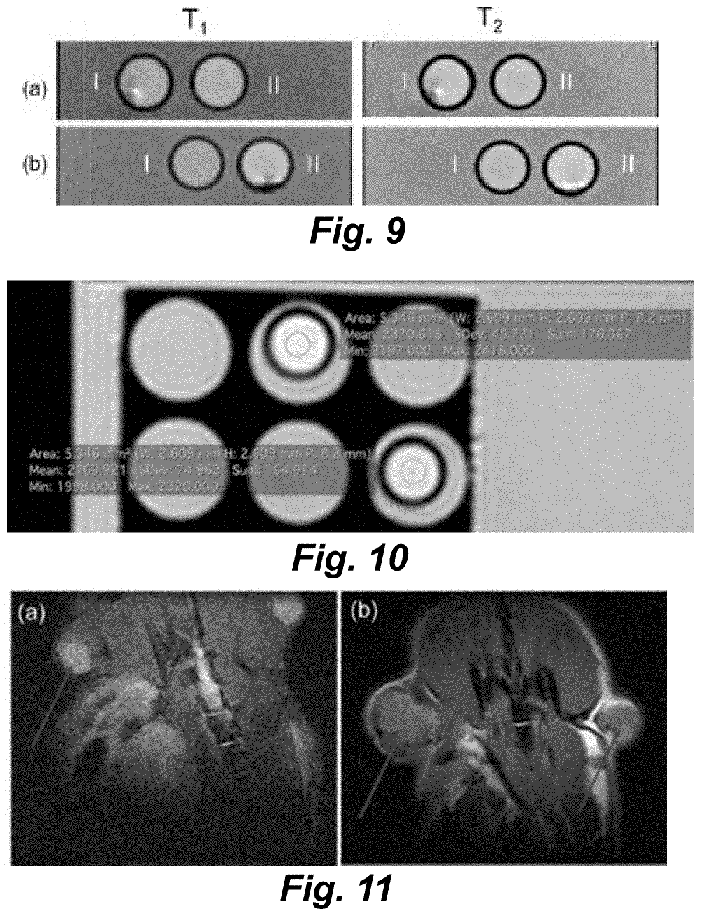

The PVPON used as the outmost layer was also shown to play a protective role for the MR imaging activity. When the (TA/PVPON)₆(Fe₂O₃/PVPON)₂ capsules were incubated in 100% fetal bovine serum (FBS) at 37° C. for 24 hours, their MRI contrast during and after the incubation was compared with that of the capsules which were never exposed to FBS (FIG. 9). The capsules MR-imaged in the presence of FBS did not show any significant change in contrast in either mode. After 24 hours, the FBS was removed from the capsule suspension by rinsing the capsules with buffer at pH=7.4, using centrifugation. The contrast intensity of the FBS treated capsules was the same as that of the capsules without any FBS treatment, which demonstrates that under relevant biological conditions the (TA/PVPON/Fe₂O₃) capsule system can remain stable and that the Fe₂O₃ NPs are not leached from within the capsule shell (FIG. 9).

### Example 21

MR imaging contrast from the capsules in vivo: To explore the clinical MR imaging potential of drug loaded (TA/PVPON/Fe₂O₃) systems, the capsules were loaded with DOX (˜0.8 pg DOX per capsule) and injected in mice followed by in vivo imaging at time points of 5 min, 4 h, and 48 h post injection. FIGS. 5A-5C show 3 T MRI images of the mice (20-25 g weight) 5 min (FIG. 5A) and 4 h (FIG. 5B) after tail vein injection with DOX-loaded (TA/PVPON)₆(Fe₂O₃/PVPON)₂ capsules (top image is T₁-weighted). Transient cortical enhancement in the kidney can be seen 5 min post-injection (FIG. 5A).

The loss of sustained contrast in the kidney after 4 h (FIG. 5B) suggests that the capsules do not localize permanently in the kidney and can transit along the blood stream to other locations. Indeed, the MRI image in FIG. 5C (T₂-weighted) shows a mouse 48 h after capsule injection exhibiting contrast enhancement in the heart (FIG. 5C, right) unlike the control mouse (capsule-free) with no MRI brightness in the heart area (FIG. 5C, left).

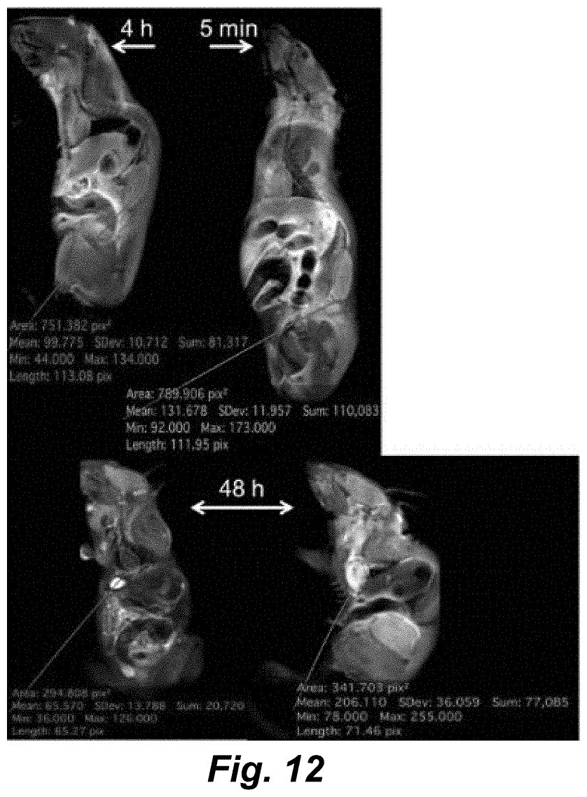

ROI analyses shown in FIG. 12 show that a significant difference in tissue contrast (P<0.0001) can be observed at the different time points, which can indicate that the capsules are not localized in any of the tissues for an extended period of time. The images shown here were taken immediately post mortem to “freeze” the circulation time point and clarify the circulation behavior. This coincidentally eliminates the flow-void effect which can cause loss of imaging contrast in the heart for MR imaging. These data suggest that the DOX-loaded (TA/PVPON)₆(Fe₂O₃/PVPON)₂ capsules can circulate continuously for at least 48 h; contrast enhancement is observed in the heart and kidneys even 48 h after injection. Importantly, incorporation of Fe₂O₃ NPs within the (TA/PVPON) capsule shell prevented the quick post-injection accumulation in the bladder observed for free Fe₂O₃ NPs as reported previously (Sherwood et al., (2017) Nanoscale 9: 11785). The extended circulation time of the (TA/PVPON) capsules may be attributed to their elasticity (0.6 MPa) (Lisunova et al., (2011) Langmuir 27: 11157) and the high hydrophilicity of PVPON which can prevent protein adsorption.

### Example 22

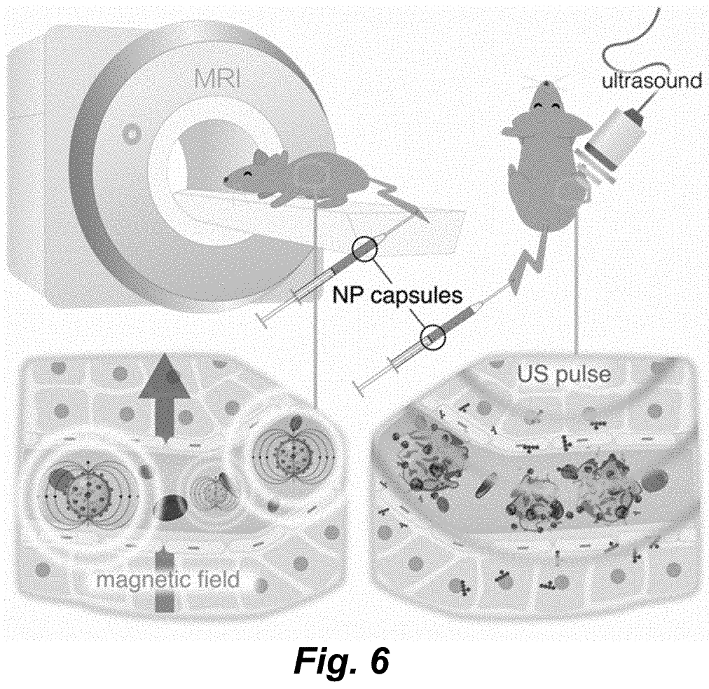

Release of DOX from (TA/PVPON)₆(Fe₂O₃/PVPON)₂ capsules in vivo triggered by focused ultrasound in vivo: DOX can be delivered from (TA/PVPON)₆(Fe₂O₃/PVPON)₂ capsules to a selected bilateral flank tumor in mice via HIFU irradiation (FIG. 6). DOX-loaded capsules (2×10⁸ capsules mL⁻¹) were injected along with the cavitation enhancer Definity (30 μL kg⁻¹) through the tail vein and ultrasound was applied to one of the two tumors (+US) for 2 min post-injection (1.0 MHz HIFU, 750 mVp/p 10 ms bursts at a 1 Hz repetition rate (1% duty cycle) for 120 s). After 15 min, the mice were euthanized and the tumors and four organ tissues (lung, liver, kidney, and spleen) were harvested. As seen in the fluorescent images of the tumors (FIGS. 7B and 7D), although DOX was present in both control (−US) and ultrasound-treated tumors, ultrasound application resulted in a 42% increase in the mean DOX fluorescent intensity as analyzed using an IVIS Lumina III fluorescence imaging device (FIG. 7B).

Histological analysis of the tumor tissues also revealed the presence of DOX fluorescence in the ultrasound -untreated (FIG. 8A, panels (a) and (c)) and ultrasound-treated (FIG. 8A, panels (b) and (d)) tumor sections. While the DOX fluorescence present in the untreated tumor suggests that the capsules can extravasate into the tumors due to the leaky cancerous vasculature, the increased fluorescence seen in the ultrasound-treated tumors suggests that the loaded drug was released into the targeted tissue.

DOX quantification in the off-target organs and ultrasound-treated tumors using HPLC-MS showed that the majority of DOX release (1809±460 ng mL⁻¹) occurred in the ultrasound-treated tumor (FIG. 7C) with small amounts of DOX in the off-target lung, liver, kidney, and spleen. The corresponding amounts of released DOX of 106±8 ng mL⁻¹ in the spleen, 135±14 ng mL⁻¹ in the liver, 92±18 ng mL⁻¹ in the kidney, and 116±29 ng mL⁻¹ in the lung, which range from 7% (liver) to 5% (kidney) of the DOX released in the target tumor tissues, respectively, demonstrates that the release of doxorubicin was highly localized to the site of ultrasound treatment at the tumor. Indeed, the data shows a 16-fold increase in DOX localization for the tumors that were treated with the focused ultrasound compared to the off-target organs.

To reinforce that the increased fluorescence in the ultrasound-treated tumor was due to released DOX, the amount of iron per gram of tissue lysates of both ultrasound-treated and ultrasound-untreated tumors was quantified using NMR relaxometry (Sherwood et al., (2017) AIP Advances 7: 056728). There was a nonsignificant difference in the iron content of the bilateral tumors with 6.3±3.0 μg Fe per gram of tumor in untreated and 7.3±3.7 μg Fe per gram of tumor in ultrasound-treated tumors (FIG. 8B), which suggests that similar amounts of Fe-containing capsules extravasated into the control and ultrasound-treated tumors. Therefore, the increased DOX concentration in the ultrasound-treated tumor can be attributed to DOX released from the capsules by the ultrasound treatment.

What is claimed:

1. A composition comprising a layer-by-layer plurality of polymer bilayers, wherein each polymer bilayer comprises a polymer layer hydrogen-bonded to a polyphenolic tannin layer, and wherein at least one of the bilayers further comprises a plurality of iron oxide nanoparticles attached thereto.

2. The composition of claim 1, wherein the composition comprises from 1 to about 20 polymer bilayers.

3. The composition of claim 1, wherein the composition comprises 6 polymer bilayers.

4. The composition of claim 1, wherein the polymer layer of each bilayer is a poly(N-vinylpyrrolidone) layer.

5. The composition of claim 1, wherein the iron oxide nanoparticles comprise ferric oxide and tannic acid.

6. The composition of claim 1, wherein the plurality of iron oxide nanoparticles are attached to at least one polymer layer.

7. The composition of claim 1, wherein the at least one polymer layer having the iron oxide nanoparticles attached thereto is a poly(N-vinylpyrrolidone) layer.

8. The composition of claim 1, wherein the composition is as a capsule defining an internal volume.

9. The composition of claim 1, wherein the layer-by-layer composition is deposited as a capsule encapsulating a core substrate.

10. The composition of claim 1, wherein the core substrate is in contact with a polyphenolic tannic acid layer of a bilayer.

11. The composition of claim 8, further comprising a plurality of poly(N-vinylpyrrolidone) layers, each of said poly(N-vinylpyrrolidone) layers alternating with a layer of iron oxide-tannic acid nanoparticles.

12. The composition of claim 8, further comprising an outer poly(N-vinylpyrrolidone) layer encapsulating the layer-by-layer composition.

13. The composition of claim 12, wherein the outer poly(N-vinylpyrrolidone) layer encapsulating the layer-by-layer composition comprises a functional moiety attached thereto.

14. The composition of claim 13, wherein the functional moiety being selected from the group consisting of: a detectable moiety, an immunomodulatory molecule, a growth factor, a cell receptor ligand, a polypeptide cell receptor, or any combination thereof.

15. The composition of claim 9, wherein the core substrate comprises at least one pharmacologically active agent.

16. The composition of claim 8, wherein the composition encapsulates at least one pharmacologically active agent within the internal volume.

17. The composition of claim 6, wherein the core substrate is removable.

18. A capsule, wherein the capsule comprises a wall encapsulating a pharmacologically active agent, wherein the wall of the capsule comprises:
a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer;
a plurality of poly(N-vinylpyrrolidone) layers, each of said poly(N-vinylpyrrolidone) layers alternating with a layer of iron oxide-tannic acid nanoparticles; and
an outer poly(N-vinylpyrrolidone) layer.

19. The composition of claim 18, wherein the outer poly(N-vinylpyrrolidone) layer comprises a functional moiety attached thereto, the functional moiety being selected from the group consisting of: a detectable moiety, an immunomodulatory molecule, a growth factor, a cell receptor ligand, a polypeptide cell receptor, or any combination thereof.

20. The composition of claim 18, wherein the capsule is mixed with a pharmaceutically acceptable carrier.

21. A method of generating a layer-by layer composition, wherein said layer-by layer composition comprises an MRI contrast agent and a pharmacologically active composition, the method comprising the steps of:
(a) obtaining a silica core substrate particle comprising a pharmacologically active agent;
(b) obtaining a population of tannic acid-modified iron-oxide nanoparticles;
(c) contacting the porous silica core of step (a) with a solution of a cationic polymer, thereby coating the porous silica core particle with the cationic polymer;
(d) encapsulating the porous silica core particle of step (c) by depositing thereon a capsule comprising a layer-by-layer polymer coating, wherein said polymer coating comprises a plurality of tannic acid-poly(N-vinylpyrrolidone)-bilayers, wherein the tannic acid layer of a first bilayer is in contact with the porous silica core;
(e) depositing a plurality of tannic acid-modified iron-oxide nanoparticles on a poly(N-vinylpyrrolidone) layer of a bilayer;
(f) depositing a plurality of alternating poly(N-vinylpyrrolidone)-tannic acid-modified iron-oxide nanoparticle layers on the surface of the product of step (e);
(g) depositing an outer poly(N-vinylpyrrolidone) layer on the surface of the product of step (f); and
(h) removing the silica core from the capsule while leaving the pharmacologically active agent within the capsule.

22. The method of claim 21, further comprising the step of attaching a functional moiety to the outer poly(N-vinylpyrrolidone) layer.

22. The method of claim 21, wherein the functional moiety is selected from the group consisting of: a detectable moiety, an immunomodulatory molecule, a growth factor, a cell receptor ligand, a polypeptide cell receptor, or any combination thereof.

23. A method of delivering a pharmacologically active agent to a patient in need thereof, the method comprising the steps:
(a) administering to a patient a pharmacologically active composition comprising a capsule, wherein the capsule comprises a wall encapsulating a pharmacologically active agent, wherein the wall of the capsule comprises:
a layer-by-layer plurality of polymer bilayers, each polymer bilayer comprising a poly(N-vinylpyrrolidone) layer hydrogen-bonded to a polyphenolic tannin layer, wherein at least one of the bilayers further comprises a plurality of iron oxide-tannic acid nanoparticles attached to the poly(N-vinylpyrrolidone) layer of the bilayer;
a plurality of poly(N-vinylpyrrolidone) layers, each of said poly(N-vinylpyrrolidone) layers alternating with a layer of iron oxide-tannic acid nanoparticles; and
an outer poly(N-vinylpyrrolidone) layer;
(b) monitoring by magnetic resonance imaging (MRI) the delivery of the pharmacologically active composition to a selected site within the patient; and
(c) administering an ultrasound emission to the patient, wherein the ultrasound emission has a frequency and intensity that disrupts the wall of the capsule of the pharmacologically active composition within the patient, thereby releasing the pharmacologically active agent to a tissue of the selected site patient.
